(19) United States
(12) Patent Application Publication
(10) Pub. No.: US 2019/0262263 A1
(43) Pub. Date: Aug. 29, 2019

(54) COMPOSITIONS, DEVICES AND METHODS FOR THE TREATMENT OF OPIOID-RECEPTOR-MEDIATED CONDITIONS

(71) Applicants: Opiant Pharmaceuticals, Inc., Santa Monica, CA (US); Aegis Therapeutics, LLC, San Diego, CA (US)

(72) Inventors: Roger Crystal, Santa Monica, CA (US); Edward T. Maggio, San Diego, CA (US)

(21) Appl. No.: 16/348,031

(22) PCT Filed: Nov. 9, 2017
(86) PCT No.: PCT/US2017/060963

§ 371 Date: May 7, 2019

Related U.S. Application Data
(60) Provisional application No. 62/525,057, filed on Jun. 26, 2017.
(60) Provisional application No. 62/419,743, filed on Nov. 9, 2016.

(51) Int. Cl. A61K 9/00; A61P 25/36; A61K 31/485; A61K 9/08; A61K 47/02; A61K 47/18; A61K 47/26

(52) U.S. Cl. CPC A61K 9/0043; A61P 25/36; A61K 31/485; A61K 47/26; A61K 47/02; A61K 47/183; A61K 47/186; A61K 9/08

(57) ABSTRACT

Drug products adapted for nasal delivery comprising naltrexone, alone or in combination with excipients, are provided. Pre-primed devices for intranasal administration of the drug products are also provided. In addition, methods for treating and preventing a variety of opioid receptor-mediated diseases, disorders, addictions, symptoms, reward-based behaviors, and conditions with the drugs products are provided.

### CROSS REFERENCE TO RELATED APPLICATIONS

This application claims the benefit of U.S. Provisional Application Ser. No. 62/419,743, entitled “Compositions and Methods for the Treatment of Opioid-Overdose and Opioid-Receptor-Mediated Conditions,” filed Nov. 9, 2016, and U.S. Provisional Application Ser. No. 62/525,057, entitled “Compositions, Devices, and Methods for the Treatment of Opioid-Overdose and Opioid-Receptor-Mediated Conditions,” file Jun. 26, 2017, the entire contents of which are incorporated herein by reference in their entireties for all purposes.

The present invention related generally to pharmaceutical compositions and more particularly to pharmaceutical compositions including an opioid antagonist and methods of use thereof. Opioid receptors are G protein-coupled receptors (GPCRs) activated both by endogenous opioid peptides and by clinically important alkaloid analgesic drugs, such as morphine. The three principal types of opioid receptors are the δ-opioid receptor, the κ-opioid receptor, and the μ-opioid receptor. Opioids (i.e., opioid agonists or opioid receptor agonists) depress respiration, which is controlled principally through medullary respiratory centers with peripheral input from chemoreceptors and other sources. Opioids produce inhibition at the chemoreceptors via μ-opioid receptors and in the medulla via μ- and δ-opioid receptors. While many neurotransmitters mediate the control of respiration, glutamate and γ-aminobutyric acid (GABA) are the major excitatory and inhibitory neurotransmitters, respectively. This explains the potential for interaction of opioids with benzodiazepines and alcohol: both benzodiazepines and alcohol aid the inhibitory effect of GABA at the GABAA receptor, while alcohol also decreases the excitatory effect of glutamate at NMDA receptors.

In 2016, approximately 64,000 people died from drug overdoses. At least 14,400 of these deaths involved prescription opioid analgesics, almost 3,300 of these deaths involved methadone, 15,400 of these deaths involved heroin, and over 20,000 of these deaths were attributed to fentanyl and related synthetic opioids. Taken together, the number of opioid-related overdose deaths in 2016 far exceeded both the peak number of H.I.V. related deaths and the peak number of fatalities related to firearms, and have drastically increased over the past nine years.

Naltrexone was initially developed to treat opioid dependence due to its effect of blocking the euphoric effects of opioids. Naltrexone tablet formulations for oral administration have been used for treating opioid addiction since 1984. Long-acting depot forms of naltrexone to be administered once monthly or longer were developed to improve compliance. Data from clinical trials demonstrated that the depot formulations were effective in reducing relapse to opioid use. Currently, there is one intramuscular, extended-release formulation, and one oral formulation, of naltrexone (Vivitrol®) for monthly administration approved by the FDA. An intranasal (IN) formulation of naltrexone has the potential to be used for treating a wide variety of opioid-receptor-mediated mediated diseases, disorders, addictions, symptoms, reward-based behaviors, or conditions without the use of needles or an extended-release formulation.

There remains a need to lower the risk of opioid overdose. The methods and compositions described herein meet this and other needs. Further, there remains a need for durable, easy-to-use, needleless devices with storage-stable formulations that can enable untrained individuals to quickly deliver a therapeutically effective dose of a rapid-acting opioid antagonist to an opioid overdose patient or subject suffering from an opioid-receptor mediated disease, disorder, addiction, symptom, reward-based behavior, or condition. The therapeutically effective dose should be sufficient to obviate the need for the untrained individual to administer either a second dose of opioid antagonist or an alternative medical intervention to the patient, and to stabilize the patient until professional medical care becomes available or as necessary to treat or prevent an ongoing medical condition. The devices described herein meet this and other needs.

Provided herein are methods of treating opioid overdose or a symptom thereof, or of treating a reward-based disorder or a symptom thereof, comprising intranasally administering naltrexone. The method comprises nasally administering to a patient in need thereof a therapeutically effective amount of naltrexone or a pharmaceutically acceptable salt thereof, wherein the therapeutically effective amount is equivalent to about 2 mg to about 16 mg of naltrexone or a pharmaceutically acceptable salt thereof.

Also provided are devices adapted for nasal delivery of a pharmaceutical composition to a patient. The device comprises a therapeutically effective amount of naltrexone or a pharmaceutically acceptable salt thereof, wherein the therapeutically effective amount is equivalent to about 2 mg to about 16 mg of naltrexone or a pharmaceutically acceptable salt thereof.

Further provided are methods of treatment of addictions or disorders associated with reward-based behavior (reward based disorders), comprising intranasally administering to the subject a therapeutically effective amount of naltrexone or a pharmaceutically acceptable salt thereof.

In some embodiments, the reward-based behavior derives from the mesolimbic pathway in the brain of the subject. In some embodiments, the reward-based behavior is dopamine-mediated. In some embodiments, the addictions or disorders are chosen from alcohol, tobacco, opioids, prescription drugs, cocaine, cannabis, amphetamines, hallucinogens, inhalants, phencyclidine, kleptomania, pyromania, gambling, and eating.

In some embodiments, the IN formulation is administered prior to exposure to an addictive substance or behavior. In some embodiments, the IN formulation is administered between about 1 and about 2 hours prior to exposure to an addictive substance or behavior. In some embodiments, the IN formulation is administered daily. In some embodiments, the IN formulation is administered twice daily. In some embodiments, the IN formulation is administered three times daily. In some embodiments, the IN formulation is administered four times daily. In some embodiments, the IN formulation is administered as needed by the subject throughout the day. In some embodiments, the IN formulation is administered once daily, followed by additional, subsequent administrations as needed by the subject throughout the day. In some embodiments, the IN formulation is administered contemporaneously with exposure to an addictive substance or behavior. In some embodiments, the IN formulation is administered following exposure to an addictive substance or behavior. In some embodiments, the IN formulation is administered between about 5 minutes and about 15 minutes before exposure to an addictive substance or behavior.

In some embodiments, the IN formulation comprises an aqueous solution. In some embodiments, the IN formulation comprises about 4 mg naltrexone or a salt thereof. In some embodiments, about 0.1 mL of said formulation is delivered to the subject. In some embodiments, the formulation comprises about 40 mg/mL naltrexone or a salt thereof.

In some embodiments, the IN formulation is administered as a single administration to one nostril. In some embodiments, the IN formulation is administered as two administrations, one to each nostril. In some embodiments, the IN formulation is administered as four administrations, two to each nostril.

In some embodiments, the IN formulation comprising a therapeutically effective amount of naltrexone is administered in conjunction with an excipient. In some embodiments, the excipient is an absorption enhancer. In some embodiments, the absorption enhancer is an alkylsaccharide or alkylglycoside, such as dodecyl maltoside.

In some embodiments, the absorption enhancer is chosen from benzalkonium chloride, an alkylsaccharide, chitosan, cyclodextrins, deoxycholic acid, glycocholic acid, laureth-9, taurocholic acid, and taurodihydrofusidic acid.

In some embodiments, the IN formulation additionally comprises one or more excipients selected from sodium chloride, benzalkonium chloride, edetate disodium, and an acid. In some embodiments, the acid is sufficient to achieve a pH of about 3.5 to about 5.5.

In some embodiments, the therapeutically effective amount comprises about 2 to about 16 mg of naltrexone. In some embodiments, the therapeutically effective amount comprises about 2, about 3, about 4, about 5, about 6, about 7, about 8, about 9, about 10, about 11, about 12, about 13, about 14, about 15, or about 16 mg of naltrexone per day.

In some embodiments, the therapeutically effective amount of naltrexone is administered in 4 mg doses throughout the day as needed by the subject.

In some embodiments, the therapeutically effective amount of naltrexone is administered as a first 4 mg dose in the morning, and subsequent 4 mg doses as needed prior to exposure to an addictive substance or behavior. In some embodiments, the therapeutically effective amount of naltrexone is administered as a first 4 mg dose in the morning, and subsequent 4 mg doses as needed contemporaneously with exposure to an addictive substance or behavior. In some embodiments, the therapeutically effective amount of naltrexone is administered as a first 4 mg dose in the morning, and subsequent 4 mg doses as needed after prior to exposure to an addictive substance or behavior.

Also disclosed herein is a device adapted for nasal delivery of a pharmaceutical composition to a subject suffering from an addiction or disorder associate with reward-based behavior, comprising a therapeutically effective amount of naltrexone and pharmaceutically acceptable salts thereof. In some embodiments, the device is pre-primed. In some embodiments, the device can be primed before use. In some embodiments, the device is a single-dose device. In some embodiments, the device is a multi-dose device.

Disclosed herein is a method of achieving a plasma concentration of naltrexone therapeutically effective to treat opioid overdose in a patient in need thereof while maintaining a plasma concentration of 6β-naltrexol below about 4 ng/Ml. The method comprises the intranasal administration of a pharmaceutical formulation comprising between about 2 mg and about 16 mg naltrexone or a salt or hydrate thereof.

Disclosed herein is a method of treating a reward-based disorder in a subject with 40 minutes administration of an intranasal pharmaceutical formulation. The formulation comprises between about 2 mg and about 12 mg naltrexone and between about 0.05% and about 2.5% (w/v) dodecyl maltoside.

Disclosed herein is a method of treating a reward based disorder in a patient for at least 2 hours, comprising the administration of an intranasal pharmaceutical formulation, the formulation comprising between about 2 mg and about 12 mg naltrexone and between about 0.05% and about 2.5% (w/v) dodecyl maltoside.

Also disclosed herein is an intranasal pharmaceutical formulation comprising naltrexone that achieves a Cₘₐₓ of at least 5 ng/mL within 40 minutes.

### BRIEF DESCRIPTION OF THE DRAWINGS

FIG. 1 is a graphical plot showing the mean naltrexone concentration in ng/mL (logarithmic scale) versus time in hours following single administration of 4 mg plus Intravail® intranasal (large circles), 50 mg oral (small upright triangles), 4 mg intranasal (small circles), and 2 mg intramuscular (large upside-down triangles).

FIG. 2 is a graphical plot showing the mean 6β-naltrexol concentration in ng/mL (logarithmic scale) versus time in hours following single administration of 4 mg plus Intravail® intranasal (large circles), 50 mg oral (small upright triangles), 4 mg intranasal (small circles), and 2 mg intramuscular (large upside-down triangles).

### DETAILED DESCRIPTION

Disclosed herein are methods and compositions for the treatment of opioid overdose, comprising administering an intranasal formulation of an opioid antagonist. Also disclosed herein are methods and compositions for the treatment of opioid-receptor-mediated-diseases, disorders, addictions, symptoms, reward-based behaviors, or conditions, comprising administering an intranasal formulation of an opioid antagonist alone or in combination with an absorption enhancer. For clarity and consistency, the following definitions will be used throughout this patent document.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

As used herein, the term “absorption enhancer” refers to a functional excipient included in formulations to improve the absorption of a pharmacologically active drug. This term usually refers to an agent whose function is to increase absorption by enhancing membrane permeation, rather than increasing solubility. As such, such agents are sometimes called permeation enhancers. In particular, absorption enhancers described herein may improve paracellular transport (i.e., passage through intercellular spaces and tight junctions), transcellular transport (i.e., passive diffusion or active transport across cellular membranes), or transcytosis (i.e., cellular vesicular uptake). Ozsoy et al., Molecules 14:3754-79, 2009.

Examples of absorption enhancers include aprotinin, benzalkonium chloride, benzyl alcohol, capric acid, ceramides, cetylpyridinium chloride, chitosan, cyclodextrins, deoxycholic acid, decanoyl carnitine, EDTA, glycocholic acid, glycodeoxycholic acid, glycofurol, glycosylated sphingosines, glycyrrhetinic acids, 2-hydroxypropyl-β-cyclodextrin, laureth-9, lauric acid, lauroyl carnitine, sodium lauryl sulfate, lysophosphatidylcholine, menthol, poloxamer 407 or F68, poly-L-arginine, polyoxyethylene-9-lauryl ether, polysorbate 80, propylene glycol, quillaia saponin, salicylic acid, sodium salt, β-sitosterol-β-D-glucoside, sucrose cocoate, taurocholic acid, taurodeoxycholic acid, taurodihydrofusidic acid, and alkylsaccharides, including but not limited to dodecyl maltoside, dodecyl-β-D-maltoside, tetradecyl maltoside, tetradecyl-β-D-maltoside and sucrose dodecanoate. Alkylsaccharides (e.g., nonionic alkylsaccharide surfactants such as alkylglycosides and sucrose esters of fatty acids that consist of an aliphatic hydrocarbon chain coupled to a sugar moiety by a glycosidic or ester bond, respectively), cyclodextrins (cyclic oligosaccharides composed of six or more monosaccharide units with a central cavity, which form inclusion complexes with hydrophobic molecules and they have primarily been used to increase drug solubility and dissolution and to enhance low molecular weight drug absorption), chitosans (linear cationic polysaccharides produced from the deacetylation of chitin), and bile salts and their derivatives (such as sodium glycocholate, sodium taurocholate, and sodium taurodihydrofusidate) tend to be amongst the best-tolerated absorption enhancers. See, e.g., Aungst, AAPS Journal 14(1):10-8, 2011; and Maggio, J. Excipients and Food Chem. 5(2):100-12, 2014.

As used herein, the term “alkylsaccharide” refers to an absorption enhancer. As used herein, an alkylsaccharide refers to any sugar joined by a linkage to any hydrophobic alkyl, as is known in the art. Alkylsaccharides of the present invention can include, but are not limited to: alkylsaccharides, such as octyl-, nonyl-, decyl-, undecyl-, dodecyl-, tridecyl-, tetradecyl-, pentadecyl-, hexadecyl-, heptadecyl-, and octadecyl-α- or β-D-maltoside, -glucoside or -sucroside; alkyl thiomaltosides, such as heptyl, octyl, dodecyl-, tridecyl-, and tetradecyl-β-D-thiomaltoside; alkyl thioglucosides, such as heptyl- or octyl 1-thio α- or β-D-glucopyranoside; alkyl thiosucroses; alkyl maltotriosides; long chain aliphatic carbonic acid amides of sucrose β-amino-alkyl ethers; derivatives of palatinose and isomaltamine linked by amide linkage to an alkyl chain; derivatives of isomaltamine linked by urea to an alkyl chain; long chain aliphatic carbonic acid ureides of sucrose β-amino-alkyl ethers; and long chain aliphatic carbonic acid amides of sucrose β-amino-alkyl ethers. The hydrophobic alkyl can be chosen of any desired size, depending on the hydrophobicity desired and the hydrophilicity of the saccharide moiety. For example, one preferred range of alkyl chains is from about 9 to about 24 carbon atoms. An even more preferred range is from about 9 to about 16 or about 14 carbon atoms. Similarly, some preferred saccharides include maltose, sucrose, and glucose linked by glycosidic linkage to an alkyl chain of 9, 10, 12, 13, 14, 16, 18, 20, 22, or 24 carbon atoms, e.g., nonyl-, decyl-, dodecyl- and tetradecyl sucroside, glucoside, and maltoside, etc.

As use herein, a “saccharide” is inclusive of monosaccharides, oligosaccharides or polysaccharides in straight chain or ring forms, or a combination thereof to form a saccharide chain. Oligosaccharides are saccharides having two or more monosaccharide residues. The saccharide can be chosen, for example, from any currently commercially available saccharide species or can be synthesized. Some examples of the many possible saccharides to use include glucose, maltose, maltotriose, maltotetraose, sucrose and trehalose. Preferable saccharides include maltose, sucrose and glucose.

As used herein, the term “active ingredient” or “pharmaceutically active compound” is defined in the context of a “pharmaceutical composition” and is intended to mean a component of a pharmaceutical composition that provides the primary pharmacological effect, as opposed to an “inactive ingredient” which would generally be recognized as providing no pharmaceutical benefit.

As used herein, the term “actuation” refers to operation of the drug delivery device such that the pharmaceutical composition is delivered therefrom.

The term “addiction,” as used herein, refers to a medical condition characterized by compulsive engagement in rewarding stimuli despite adverse consequences. The term, “addictive behavior,” as used herein, refers to a behavior that is both rewarding and reinforcing. The term “reinforcing stimuli,” as used herein refers to stimuli that increase the probability of repeating behaviors paired with them. The term, “rewarding stimuli,” as used herein, refers to stimuli that the brain interprets as intrinsically positive or as something to be approached.

As used herein, the term “agonist” refers to a moiety that interacts with and activates a receptor, and thereby initiates a physiological or pharmacological response characteristic of that receptor. As used herein, the term “antagonist” refers to a moiety that competitively binds to a receptor at the same site as an agonist (for example, the endogenous ligand), but which does not activate the intracellular response initiated by the active form of the receptor and can thereby inhibit the intracellular responses by an agonist or partial agonist. An antagonist does not diminish the baseline intracellular response in the absence of an agonist or partial agonist. The term “inverse agonist” refers to a moiety that binds to the endogenous form of the receptor or to the constitutively activated form of the receptor and which inhibits the baseline intracellular response initiated by the active form of the receptor below the normal base level of activity which is observed in the absence of an agonist or partial agonist.

As used herein, the term “antimicrobial preservative” refers to a pharmaceutically acceptable excipient with antimicrobial properties which is added to a pharmaceutical composition to maintain microbiological stability.

As used herein, the term “application” refers to a program executed by a computer.

As used herein, the term “AUC” refers to the area under the drug plasma concentration-time curve. As used herein, the term “AUC₀₋ₜ” refers to the area under the drug plasma concentration-time curve from t=0 to the last measurable concentration. As used herein, the term “AUC_{0-∞}” refers to the area under the drug plasma concentration-time curve extrapolated to Go. As used herein, the term “AUC_{0-t/D}” refers to the AUC₀₋ₜ normalized to 0.4 mg IM naltrexone. As used herein, the term “AUC_{0-∞/D}” refers to the AUC_{0-∞} normalized to 0.4 mg IM naltrexone

As used herein, the term “bioavailability (F)” refers to the fraction of a dose of drug that is absorbed from its site of administration and reaches, in an unchanged form, the systemic circulation. As used herein, the term “absolute bioavailability” is used when the fraction of absorbed drug is related to its IV bioavailability. It may be calculated using the following formula:

$$F = \frac{{AUC}_{extravascular}}{{AUC}_{intravenous}} \times \frac{{Dose}_{intravenous}}{{Dose}_{extravascular}}$$

The term relative bioavailability (Fᵣₑₗ) is used to compare two different extravascular routes of drug administration and it may be calculated using the following formula:

$${F}_{rel} = \frac{{AUC}_{extravascular 1}}{{AUC}_{extravascular 2}} \times \frac{{Dose}_{extravascular 2}}{{Dose}_{extravascular 1}}$$

As used herein, the term “clearance (CL)” refers to the rate at which a drug is eliminated divided by its plasma concentration, giving a volume of plasma from which drug is completely removed per unit of time. CL is equal to the elimination rate constant (λ) multiplied by the volume of distribution (V_{d}), wherein “V_{d}” is the fluid volume that would be required to contain the amount of drug present in the body at the same concentration as in the plasma. As used herein, the term “apparent clearance (CL/F)” refers to clearance that does not consider the bioavailability of the drug. It is the ratio of the dose over the AUC.

As used herein, the term “Cₘₐₓ^{”} refers to the maximum observed plasma concentration. As used herein, the term “Cₘₐₓ/D” refers to Cₘₐₓ normalized to 0.4 mg IM naltrexone.

As used herein, the term “coefficient of variation (CV)” refers to the ratio of the sample standard deviation to the sample mean. It is often expressed as a percentage.

As used herein, the term “confidence interval” (“CI”) refers to a range of values which will include the true average value of a parameter a specified percentage of the time.

As used herein, the term “drug delivery device,” refers to an apparatus capable of delivering a drug to patient in need thereof.

The term “delivery time,” refers to the amount of time that elapses between a determination made by a healthcare professional, or an untrained individual that an individual is in need of nasal delivery of an opioid antagonist and completion of the delivery.

The term “disease” is generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “elimination rate constant (2)” refers to the fractional rate of drug removal from the body. This rate is constant in first-order kinetics and is independent of drug concentration in the body. λ is the slope of the plasma concentration-time line (on a logarithmic y scale). The term “λ_{z},” as used herein, refers to the terminal phase elimination rate constant, wherein the “terminal phase” of the drug plasma concentration-time curve is a straight line when plotted on a semilogarithmic graph. The terminal phase is often called the “elimination phase” because the primary mechanism for decreasing drug concentration during the terminal phase is drug elimination from the body. The distinguishing characteristic of the terminal elimination phase is that the relative proportion of drug in the plasma and peripheral volumes of distribution remains constant. During this “terminal phase” drug returns from the rapid and slow distribution volumes to the plasma, and is permanently removed from the plasma by metabolism or renal excretion.

As used herein, the term “equivalent” refers to a weight of an opioid antagonist selected from naltrexone and pharmaceutically acceptable salts thereof that is equimolar to a specified weight of naltrexone hydrochloride.

As used herein, the term “excipient” refers to a natural or synthetic substance formulated alongside the active ingredient of a medication, included for long-term stabilization, bulking up solid formulations, or to confer a therapeutic enhancement on the active ingredient in the final dosage form, such as facilitating drug absorption, reducing viscosity, or enhancing solubility.

The term “filled” refers to an association between a device and a pharmaceutical composition, for example, when a pharmaceutical composition described herein comprising a therapeutically effective amount of an opioid antagonist is present within a reservoir that forms a part of a device described herein.

As used herein, the term “hydrate” refers to an opioid antagonist described herein or a salt thereof that further includes a stoichiometric or non-stoichiometric amount of water bound by non-covalent intermolecular forces.

As used herein, the term “in need of treatment” and the term “in need thereof” when referring to treatment are used interchangeably and refer to a judgment made by a caregiver (e.g., physician, nurse, nurse practitioner, that a patient will benefit from treatment.

An individual “who is at risk for opioid overdose” includes an individual who illicitly uses opioids, on individual who accidentally ingests opioids, and an individual at risk for accidental misuse of opioids during medical opioid therapy.

As used herein, two embodiments are “mutually exclusive” when one is defined to be something which is different than the other. For example, an embodiment wherein the amount of naltrexone hydrochloride is specified to be 4 mg is mutually exclusive with an embodiment wherein the amount of naltrexone hydrochloride is specified to be 2 mg. However, an embodiment wherein the amount of naltrexone hydrochloride is specified to be 4 mg is not mutually exclusive with an embodiment in which less than about 10% of said pharmaceutical composition leaves the nasal cavity via drainage into the nasopharynx or externally.

The term “naloxone,” as used herein, refers to a compound of the following structure:

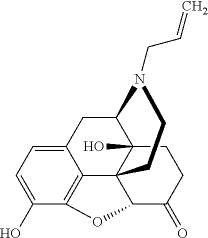

or a pharmaceutically acceptable salt, hydrate, or solvate thereof. The CAS registry number for naloxone is 465-65-6. Other names for naloxone include: 17-allyl-4,5a-epoxy-3,14-dihydroxymorphinan-6-one; (−)-17-allyl-4,5α-epoxy-3,14-dihydroxymorphinan-6-one; 4,5a-epoxy-3,14-dihydroxy-17-(2-propenyl)morphinan-6-one; and (−)-12-allyl-7,7a,8,9-tetrahydro-3,7a-dihydroxy-4aH-8,9c-iminoethanophenanthro[4,5-bcd]furan-5(6H)-one. Naloxone hydrochloride may be anhydrous (CAS Reg. No. 357-08-4) and also forms a dihydrate (CAS No. 51481-60-8). It has been sold under various brand names including Narcan®, Nalone®, Nalossone®, Naloxona®, Naloxonum®, Narcanti®, and Narcon®.

The term “naltrexone,” as used herein, refers to a compound of the following structure:

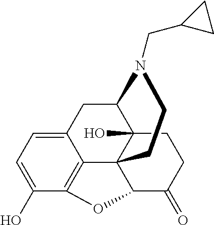

or a pharmaceutically acceptable salt, hydrate, or solvate thereof. The CAS registry number for naltrexone is 16590-41-3. Other names for naltrexone include: 17-(cyclopropylmethyl)-4,5α-epoxy-3,14-dihydroxymorphinan-6-one; (5α)-17-(cyclopropylmethyl)-3,14-dihydroxy-4,5-epoxymorphinan-6-one; and (1S,5R,13R,17S)-4-(cyclopropylmethyl)-10,17-dihydroxy-12-oxa-4-azapentacyclo[9.6.1.01,13.05,17.07,18]octadeca-7(18),8,10-trien-14-one. Naltrexone hydrochloride (CAS Reg. No. 16676-29-2) has been marketed under the trade names Antaxone®, Depade®, Nalorex®, Revia®, Trexan®, Vivitrex®, and Vivitrol®.

The term “methylnaltrexone,” as used herein, refers to a pharmaceutically acceptable salt comprising the cation (5α)-17-(cyclopropylmethyl)-3,14-dihydroxy-17-methyl-4,5-epoxymorphinanium-17-ium-6-one a compound of the following structure:

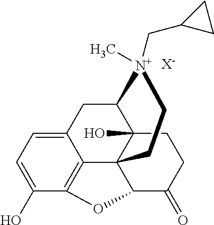

wherein X⁻ is a pharmaceutically acceptable anion. Methylnaltrexone bromide (CAS Reg. No. 75232-52-7) has been marketed under the trade name Relistor®.
The term “nalmefene,” as used herein, refers to 17-cyclopropylmethyl-4,5α-epoxy-6-methylenemorphinan-3,14-diol, a compound of the following structure:

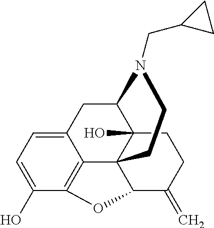

Nalmefene hydrochloride (CAS Reg. No. 58895-64-0) has been marketed under the trade names Nalmetrene®, Cervene®, Revex®, Arthrene®, and Incystene®.

As used herein, the term “nostril” is synonymous with “naris.”

As used herein, the term “opioid antagonist” includes naltrexone and pharmaceutically acceptable salts thereof. In some embodiments, the opioid antagonist is naloxone hydrochloride. In some embodiments, the opioid antagonist is naltrexone hydrochloride. In some embodiments, the nasally administering is accomplished using a device described herein.

As used herein, the term “opioid overdose” refers to an acute medical condition induced by excessive use of one or more opioids. Symptoms of opioid overdose include including respiratory depression (including postoperative opioid respiratory depression, acute lung injury, and aspiration pneumonia), central nervous system depression (which may include sedation, altered level consciousness, miotic (constricted) pupils), and cardiovascular depression (which may include hypoxemia and hypotension). Visible signs of opioid overdose or suspected opioid overdose include: unresponsiveness and/or loss of consciousness (won't respond to stimuli such as shouting, shaking, or rubbing knuckles on sternum); slow, erratic, or stopped breathing; slow, erratic, or stopped pulse; deep snoring or choking/gurgling sounds; blue or purple fingernails or lips; pale and/or clammy face; slack or limp muscle tone; contracted pupils; and vomiting. Because opioid overdose may be difficult to diagnose and/or quantify, particularly by a lay person, as used herein, treatment of opioid overdose is meant to include treatment of suspected opioid overdose in opioid-intoxicated patients. Opioids that may induce overdose include, codeine, morphine, methadone, fentanyl, oxycodone HCl, hydrocodone bitartrate, hydromorphone, oxymorphone, meperidine, propoxyphene, opium, heroin, tramadol, tapentadol, and certain narcotic-antagonist analgesics, such as, nalbuphine, pentazocine and butorphanol. In some embodiments, the opioid agonist is in a tamper-proof formulation. In some embodiments, the opioid agonist is in a tamper-resistant formulation. In some embodiments, the opioid agonist is selected from Acurox® Oxycodone DETERx®, Egalet™ hydrocodone, Egalet™ morphine, Egalet™ oxycodone, Exalgo®, Opana®, and Remoxy®.

As used herein, the term “patient” refers to any subject (preferably human) afflicted with a condition likely to benefit from a treatment with a therapeutically effective amount of an opioid antagonist.

As used herein, the term “pharmaceutical composition” refers to a composition comprising at least one active ingredient; including but not limited to, salts, solvates and hydrates of the opioid antagonists described herein, whereby the composition is amenable to use for a specified, efficacious outcome in a mammal (for example, without limitation, a human). In some embodiments, the opioid antagonist is naltrexone.

As used herein, the term “pre-primed” refers to a drug delivery device, such as a nasal spray which can deliver a pharmaceutical composition to a patient in need thereof with the first actuation of the spray pump, i.e., without the need to prime the pump prior to dosing, such as by actuating the pump one or more times until a spray appears.

As used herein, the term “prone” refers to a patient who is lying face down.

As used herein, the term “protective packaging” refers to overwrap.

The term “receptor binding or occupancy” refers to a characterization of the kinetics between a radioactive drug and receptors or other binding sites throughout the body, and characterization of the radioactive drug binding affinity to these receptors.

The term “recovery position,” as used herein, means a position of the human body in which a patient lies on his/her side, with a leg or knee out in front (e.g., to prevent rolling onto his/her stomach) and at least one hand supporting the head (e.g., to elevate the face to facilitate breathing and prevent inhalation of vomit).

As used herein, the term “providing” in the context of providing a co-packaged drug product as disclosed herein to an individual includes co-packaging the drug product, prescribing the co-packaged drug product, and dispensing the co-packaged drug product. The providing may be done either directly to an individual (for example, to an individual for whom an opioid agonist prescription is appropriate, or who is otherwise at risk of opioid overdose) or to a second individual

As used herein, the term “solvate” refers to an opioid antagonist described herein or a salt, thereof, that further includes a stoichiometric or non-stoichiometric amount of a solvent bound by non-covalent intermolecular forces. Preferred solvents are volatile, non-toxic, and/or acceptable for administration to humans in trace amounts.

As used herein, the term “sterile filling” refers methods of manufacturing the devices and pharmaceutical compositions described herein, such that the use of preservatives is not required. Sterile drug products may be produced using aseptic processing or terminal sterilization. Terminal sterilization usually involves filling and sealing product containers under high-quality environmental conditions. In an aseptic process, the drug product, container, and closure are first subjected to sterilization methods separately, as appropriate, and then brought together.

As used herein, the term “storage-stable” refers to a pharmaceutical composition in which at least about 95% to 99.5% of the active ingredient remains in an undegraded state after storage of the pharmaceutical composition at specified temperature and humidity for a specified time, for example, for 12 months at 25° C. and 60% relative humidity.

The term “subject,” as used herein, is intended to be synonymous with “patient,” and refers to any mammal (preferably human) afflicted with a condition likely to benefit from a treatment with a therapeutically effective amount of the opioid antagonist naltrexone.

As used herein, the term “substantially free of antimicrobial preservatives” is understood by one of ordinary skill in the art to described a pharmaceutical composition that comprises less than 1% w/w antimicrobial preservatives.

The term “therapeutically effective dose,” as used herein refers to a dose that is effective to decrease one or more observable symptoms of alcohol use disorder or a related disease, or to delay onset or mitigate the symptoms of a more serious condition that often follows the condition that a patient is currently experiencing. A therapeutically effective dose may, but need not necessarily, eliminate all symptoms of the disease. A therapeutically effective amount may, but need not necessarily, eliminate one, more, or all symptoms of a disease, disorder, or condition being treated. A therapeutically effective amount may also prevent disease progression or the appearance of further symptoms.

As used herein, the term “t_{1/2}” or “half-life” refers to the amount of time required for half of a drug to be eliminated from the body or the time required for a drug concentration to decline by half.

As used herein, the term “tonicity agent” refers to a compound which modifies the osmolality of a formulation, for example, to render it isotonic. Tonicity agents include, dextrose, lactose, sodium chloride, calcium chloride, magnesium chloride, sorbitol, sucrose, mannitol, trehalose, raffinose, polyethylene glycol, hydroxyethyl starch, glycine and the like.

As used herein, the term “tomography” refers to a process of imaging by sections. The images may be looked at individually, as a series of two-dimensional slices or together, as a computer-generated three-dimensional representation.

As used herein, the term “pharmaceutically acceptable” refers to a component of a pharmaceutical composition that it compatible with the other ingredients of the formulation and not overly deleterious to the recipient thereof.

As used herein, the term “substantially free of antimicrobial preservatives” is understood by one of ordinary skill in the art to described a pharmaceutical composition that may comprise less than 1% w/w antimicrobial preservatives.

As used herein, the term “therapeutically effective amount” refers to the amount of active compound or pharmaceutical agent that elicits the biological or medicinal response in a tissue, system, or individual that is being sought by a researcher, healthcare provider or individual.

As used herein, the term “Tₘₐₓ” refers to the time from administration of the pharmaceutical compositions described herein to maximum drug plasma concentration

As used herein, the term “untrained individual” refers to an individual administering to patient an opioid antagonist using a drug delivery device described herein, wherein the individual is not a healthcare professional and has received no training in the use of the drug delivery device.

### Opioid Antagonists

Opioid receptor antagonists are a well-recognized class of chemical agents. They have been described in detail in the scientific and patent literature. Naltrexone and its active metabolite 6β-naltrexol are opioid antagonists, with no agonist properties, at the μ-opioid receptor (MOR), the κ-opioid receptor (KOR), and the δ-opioid receptor (DOR). Naltrexone operates by reversibly blocking the opioid receptors thereby attenuating the effects of opioids. Without being limited by theory, naltrexone likely modulates the dopaminergic mesolimbic pathway (one of the primary centers for risk-reward analysis in the brain, and a tertiary pleasure center) which is believed to be a major center of the reward associated with addiction that all major drugs of abuse are believed to activate. The mechanism of action may be antagonism to endogenous opiates referred to as endorphins and enkephalins.

Provided are pharmaceutical compositions, devices adapted for nasal delivery of a pharmaceutical composition to a patient, kits comprising the foregoing, and methods of using the same in treatment, each comprising a therapeutically effective amount of an opioid antagonist selected from naltrexone and pharmaceutically acceptable salts thereof, and wherein the therapeutically effective amount, is equivalent to about 1 mg to about 10 mg of naltrexone hydrochloride.

In some embodiments, the therapeutically effective amount is equivalent to about 0.5 mg to about 12 mg of naltrexone hydrochloride. In some embodiments, the therapeutically effective amount is equivalent to about 0.5 mg to about 10 mg of naltrexone hydrochloride. In some embodiments, the therapeutically effective amount is equivalent to about 1 mg to about 12 mg of naltrexone hydrochloride. In some embodiments, the therapeutically effective amount is equivalent to about 1 mg to about 10 mg of naltrexone hydrochloride. In some embodiments, the therapeutically effective amount is equivalent to about 1 mg to about 9 mg of naltrexone hydrochloride. In some embodiments, the therapeutically effective amount is equivalent to about 1 mg to about 8 mg of naltrexone hydrochloride. In some embodiments, the therapeutically effective amount is equivalent to about 1 mg to about 7 mg of naltrexone hydrochloride. In some embodiments, the therapeutically effective amount is equivalent to about 1 mg to about 6 mg of naltrexone hydrochloride. In some embodiments, the therapeutically effective amount is equivalent to about 1 mg to about 5 mg of naltrexone hydrochloride. In some embodiments, the therapeutically effective amount is equivalent to about 1 mg to about 4 mg of naltrexone hydrochloride. In some embodiments, the therapeutically effective amount is equivalent to about 1 mg to about 3 mg of naltrexone hydrochloride. In some embodiments, the therapeutically effective amount is equivalent to about 1 mg to about 2 mg of naltrexone hydrochloride. In some embodiments, the therapeutically effective amount is equivalent to about 1.5 mg to about 10 mg of naltrexone hydrochloride. In some embodiments, the therapeutically effective amount is equivalent to about 2 mg to about 10 mg of naltrexone hydrochloride. In some embodiments, the therapeutically effective amount is equivalent to about 3 mg to about 10 mg of naltrexone hydrochloride. In some embodiments, the therapeutically effective amount is equivalent to about 4 mg to about 10 mg of naltrexone hydrochloride. In some embodiments, the therapeutically effective amount is equivalent to about 5 mg to about 10 mg of naltrexone hydrochloride. In some embodiments, the therapeutically effective amount is equivalent to about 2 mg to about 8 mg of naltrexone hydrochloride. In some embodiments, the therapeutically effective amount is equivalent to about 2 mg to about 6 mg of naltrexone hydrochloride. In some embodiments, the therapeutically effective amount is equivalent to about 1 mg of naltrexone hydrochloride. In some embodiments, the therapeutically effective amount is equivalent to about 1.5 mg of naltrexone hydrochloride. In some embodiments, the therapeutically effective amount is equivalent to about 2 mg of naltrexone hydrochloride. In some embodiments, the therapeutically effective amount is equivalent to about 2.5 mg of naltrexone hydrochloride. In some embodiments, the therapeutically effective amount is equivalent to about 3 mg of naltrexone hydrochloride. In some embodiments, the therapeutically effective amount is equivalent to about 3.5 mg of naltrexone hydrochloride. In some embodiments, the therapeutically effective amount is equivalent to about 4 mg of naltrexone hydrochloride. In some embodiments, the therapeutically effective amount is equivalent to about 4.5 mg of naltrexone hydrochloride. In some embodiments, the therapeutically effective amount is equivalent to about 5 mg of naltrexone hydrochloride. In some embodiments, the therapeutically effective amount is equivalent to about 6 mg of naltrexone hydrochloride. In some embodiments, the therapeutically effective amount is equivalent to about 7 mg of naltrexone hydrochloride. In some embodiments, the therapeutically effective amount is equivalent to about 8 mg of naltrexone hydrochloride. In some embodiments, the therapeutically effective amount is equivalent to about 9 mg of naltrexone hydrochloride. In some embodiments, the therapeutically effective amount is equivalent to about 10 mg of naltrexone hydrochloride. In some embodiments, the therapeutically effective amount is equivalent to about 0.5, about 0.6, about 0.7, about 0.8, about 0.9, about 1, about 1.2, about 1.4, about 1.6, about 1.8, about 2 mg, about 2.2, about 2.4, about 2.6, about 2.8, about 3, about 3.2, about 3.4, about 3.6, about 3.8, or about 4 mg of naltrexone hydrochloride. In some embodiments, the therapeutically effective amount is equivalent to about 0.5 to about 1.0, about 0.5 to about 1.5, about 1.5 to about 2.0, about 1.5 to about 2.5, about 1.5 to about 3.0 mg, about 1.5 to about 3.5, or about 1.5 to about 4.0 mg of naltrexone hydrochloride. In some embodiments, the therapeutically effective amount is equivalent to less than 10 mg of naltrexone hydrochloride. In some embodiments, the therapeutically effective amount is equivalent to less than 5 mg of naltrexone hydrochloride.

In some embodiments, the opioid antagonist is the only pharmaceutically active compound in pharmaceutical composition. In some embodiments, the opioid antagonist is naltrexone hydrochloride. In some embodiments, the opioid antagonist is anhydrous naltrexone hydrochloride.

The opioidergic reward pathways of the brain provide the pleasure drives for behaviors such as eating and reproduction, “natural rewards” involving the release of dopamine in the nucleus accumbens and frontal lobes. The same sensations of pleasure can also be produced by exogenous stimuli, including drugs such as alcohol, cocaine, methamphetamine, heroin, nicotine, marijuana, and other drugs, and by compulsive activities, such as gambling, and by other risk-taking behaviors. Only a minority of individuals become addicted to these compounds or behaviors. There are multiple factors that distinguish those who become addicted and those who do not. For example, one or more variant genes may be risk factors for these behaviors.

Naltrexone is commercially available as a hydrochloride salt. Naltrexone hydrochloride (17-(cyclopropylmethyl)-4,5α-epoxy-3,14-dihydroxymorphinan-6-one) prevents euphorigenic effects in patients addicted to opioids. It markedly blocks the physical dependence to intravenously administered opioids and motivates withdrawal from opioid dependency, but the patient does not develop tolerance or dependence to naltrexone. Naltrexone is also effective in reducing the craving for alcohol in the treatment of alcoholism, especially when combined with psychosocial therapy.

When naltrexone hydrochloride is administered intranasally, rather than orally, it has a significantly higher bioavailability. When administered orally, despite being almost completely absorbed from the gastrointestinal tract, naltrexone undergoes rapid and extensive first-pass metabolism to 6-β-naltrexol. As a result, the amount of naltrexone reaching systemic circulation is limited. In fact, the oral bioavailability of naltrexone has been reported to be as low as 5%. See, Gonzalez and Brogden, Drugs 35:192-213, 1988.

Provided herein are methods of treatment employing nasal delivery of a pharmaceutical composition to a patient, comprising a therapeutically effective amount of the opioid antagonist naltrexone. In some embodiments, the therapeutically effective amount is equivalent to about 4 to about 16 mg of naltrexone. In some embodiments, the therapeutically effective amount is equivalent to about 4, about 5, about 6, about 7, about 8, about 9, about 10, about 11, about 12, about 13, about 14, about 15, or about 16 mg of naltrexone. In some embodiments, the therapeutically effective amount is equivalent to about 4 mg of naltrexone hydrochloride. In some embodiments, the opioid antagonist is naltrexone hydrochloride. In some embodiments, the opioid antagonist is anhydrous naltrexone hydrochloride. In some embodiments, the opioid antagonist is naltrexone hydrochloride dihydrate.

### Nasal Drug Delivery Devices and Kits

Also provided herein are pharmaceutical compositions in a device adapted for nasal delivery to a subject suffering from opioid overdose; or opioid-receptor-mediated-diseases, disorders, addictions, symptoms, reward-based behaviors, or conditions, comprising administering an intranasal formulation of naltrexone. In some embodiments, the device is pre-primed. In some embodiments, the device can be primed before use. In some embodiments, the device can be actuated with one hand.

Nasal delivery is considered an attractive route for systemic drug delivery, especially when rapid absorption and effect are desired. In addition, nasal delivery may help address issues related to unpleasant taste, poor bioavailability, slow absorption, drug degradation, adverse events (AEs) in the gastrointestinal tract, and avoids first-pass metabolism and the hepatic toxicity associated with long-term oral naltrexone usage.

Liquid nasal formulations are mainly aqueous solutions, but suspensions and emulsions can also be delivered. In traditional spray pump systems, antimicrobial preservatives are typically required to maintain microbiological stability in liquid formulations.

Some emergency medical service (EMS) programs have developed a system using existing technologies of an approved drug and an existing medical device to administer the opioid antagonist naloxone intranasally, albeit in a non-FDA approved manner. This has been accomplished by using the injectable formulation (1 mg/mL) and administering 1 mL per nostril via a marketed nasal atomizer/nebulizer device. The system combines an FDA-approved naloxone injection product (with a Luer fitted tip, no needles) with a marketed, medical device called the Mucosal Atomization Device (MAD™ Nasal, Wolfe Tory Medical, Inc.). This initiative is consistent with the U.S. Needlestick Safety and Prevention Act (Public Law 106-430). The EMS programs recognize limitations of this system, one limitation being that it is not assembled and ready-to-use. Although this administration mode appears to be effective in reversing narcosis, the formulation is not concentrated for retention in the nasal cavity. The 1 mL delivery volume per nostril is larger than that generally utilized for intranasal drug administration. Therefore, there is loss of drug from the nasal cavity, due either to drainage into the nasopharynx or externally from the nasal cavity. The devices described herein are improved ready-to-use products specifically optimized, concentrated, and formulated for nasal delivery.

Metered spray pumps have dominated the nasal drug delivery market since they were introduced. The pumps typically deliver 100 μL (or other volumes in the range of 25-200 μL, and higher) per spray, and they offer high reproducibility of the emitted dose and plume geometry in in vitro tests.

Examples of standard metered spray pumps include those offered by Aptar Pharma, Inc., such as the multi-dose “classic technology platform” nasal spray devices. Such devices comprise a reservoir which holds multiple doses of the nasal spray formulation (e.g., 50, 100, 150, 200, 60, or 120 doses), a closure (e.g., screw, crimp, or snap-on), and an actuator which delivers anywhere from 45 to 1000 μL, (e.g. 50, 100, 140, 150, or 200 μL) of fluid per actuation to comprise a single dose. The actuator may be configured to count doses, deliver gel formulations, deliver in an upside-down configuration, etc.

In traditional spray pump systems, antimicrobial preservatives are typically required to maintain microbiological stability in liquid formulations. However, preservative-free systems are also available, e.g. the Advanced Preservative Free (APF) system from Aptar, which is vented, contains a filter membrane for air flow which prevents contamination, has a metal-free fluid path for oxidizing formulations, and can be used in any orientation. Additional nasal spray devices from Aptar and others are optimized with dispenser tips that prevent clogging (useful for high-viscosity and high-volatile formulations), actuators that do not need re-priming after long periods of disuse, etc.

Traditional spray pumps replace the emitted liquid with air, and preservatives are therefore required to prevent contamination. However, driven by the studies suggesting possible negative effects of preservatives (e.g., irritation of nasal mucosa), pump manufacturers have developed different spray systems that avoid the need for preservatives. These systems use a collapsible bag, a movable piston, or a compressed gas to compensate for the emitted liquid volume (www.aptar.com and www.rexam.com). The solutions with a collapsible bag and a movable piston compensating for the emitted liquid volume offer the additional advantage that they can be emitted upside down, without the risk of sucking air into the dip tube and compromising the subsequent spray. This may be useful for some products where the patients are bedridden and where a head-down application is recommended. Another method used for avoiding preservatives is that the air that replaces the emitted liquid is filtered through an aseptic air filter. In addition, some systems have a ball valve at the tip to prevent contamination of the liquid inside the applicator tip (www.aptar.com). More recently, pumps have been designed with side-actuation and introduced for delivery of fluticasone furoate for the indication of seasonal and perennial allergic rhinitis. The pump was designed with a shorter tip to avoid contact with the sensitive mucosal surfaces. New designs to reduce the need for priming and re-priming, and pumps incorporating pressure point features to improve the dose reproducibility and dose counters and lock-out mechanisms for enhanced dose control and safety are available (www.rexam.com and www.aptar.com).

Traditional, simple metered-dose spray pumps require priming and some degree of overfill to maintain dose conformity for the labeled number of doses. They are well suited for drugs to be administered daily over a prolonged duration, but due to the priming procedure and limited control of dosing, unless a specialty device is selected, they are less suited for drugs with a narrow therapeutic window, particularly if they are not used often. For expensive drugs and vaccines intended for single administration or sporadic use and where tight control of the dose and formulation is of importance, single-dose or bi-dose spray devices are preferred (www.aptar.com). A simple variant of a single-dose spray device (MAD™) is offered by LMA (LMA, Salt Lake City, Utah, USA; www.lmana.com). A nosepiece with a spray tip is fitted to a standard syringe. The liquid drug to be delivered is first drawn into the syringe and then the spray tip is fitted onto the syringe. This device has been used in academic studies to deliver, for example, a topical steroid in patients with chronic rhinosinusitis and in a vaccine study. A pre-filled device based on the same principle for one or two doses (Accuspray™, Becton Dickinson Technologies, Research Triangle Park, N.C., USA; www.bdpharma.com) is used to deliver the influenza vaccine FluMist™ (www.flumist.com), approved for both adults and children in the US market. A similar device for two doses was marketed by a Swiss company for delivery of another influenza vaccine a decade ago.

Pre-primed single- and bi-dose devices are also available, and consist of a reservoir, a piston, and a swirl chamber (see, e.g., the UDS UnitDose™ and BDS BiDose™ devices from Aptar, formerly Pfeiffer). The spray is formed when the liquid is forced out through the swirl chamber. These devices are held between the second and the third fingers with the thumb on the actuator. A pressure point mechanism incorporated in some devices secures reproducibility of the actuation force and emitted plume characteristics. Currently, marketed nasal migraine drugs like Imitrex® (www.gsk.com) and Zomig® (www.az.com; Pfeiffer/Aptar single-dose device), the marketed influenza vaccine Flu-Mist (www.flumist.com; Becton Dickinson single-dose spray device), and the intranasal formulation of naloxone for opioid overdose rescue, Narcan Nasal® (narcan.com; Adapt Pharma) are delivered with this type of device.

In certain embodiments, the 90% confidence interval for dose delivered per actuation is ±about 2%. In certain embodiments, the 95% confidence interval for dose delivered per actuation is ±about 2.5%.

Historically, intranasal administration of drugs in large volume, such as from syringes adapted with mucosal atomizer devices, has encountered difficulty due to the tendency of some of the formulation to drip back out of the nostril or down the nasopharynx. Accordingly, in certain embodiments, upon nasal delivery of said pharmaceutical composition to said patient, less than about 20% of said pharmaceutical composition leaves the nasal cavity via drainage into the nasopharynx or externally. In certain embodiments, upon nasal delivery of said pharmaceutical composition to said patient, less than about 10% of said pharmaceutical composition leaves the nasal cavity via drainage into the nasopharynx or externally. In certain embodiments, upon nasal delivery of said pharmaceutical composition to said patient, less than about 5% of said pharmaceutical composition leaves the nasal cavity via drainage into the nasopharynx or externally.

Current container closure system designs for inhalation spray drug products include both pre-metered and device-metered presentations using mechanical or power assistance and/or energy from patient inspiration for production of the spray plume. Pre-metered presentations contain previously measured doses or a dose fraction in some type of units (e.g., single or multiple blisters or other cavities) that are subsequently inserted into the device during manufacture or by the patient before use. Typical device-metered units have a reservoir containing formulation sufficient for multiple doses that are delivered as metered sprays by the device itself when activated by the patient.

A new nasal drug delivery method, which can be adapted to any type of dispersion technology for both liquids and powders, is breath-powered Bi-Directional™ technology. This concept exploits natural functional aspects of the upper airways to offer a delivery method that may overcome many of the inherent limitations of traditional nasal devices. Breath-powered Bi-Directional™ devices consist of a mouthpiece and a sealing nosepiece with an optimized frusto-conical shape and comfortable surface that mechanically expands the first part of the nasal valve. The user slides a sealing nosepiece into one nostril until it forms a seal with the flexible soft tissue of the nostril opening, at which point, it mechanically expands the narrow slit-shaped part of the nasal triangular valve. The user then exhales through an attached mouthpiece. When exhaling into the mouthpiece against the resistance of the device, the soft palate (or velum) is automatically elevated by the positive oropharyngeal pressure, isolating the nasal cavity from the rest of the respiratory system. This mechanism enables release of liquid or powder particles into an air stream that enters one nostril, passes entirely around the nasal septum, and exits through the opposite nostril.

With sterile filling, the use of preservatives is not required in devices, but overfill is required resulting in a waste fraction similar to the metered-dose, multi-dose sprays. To emit 100 μL, a volume of 125 μL is filled in the device (Pfeiffer/Aptar single-dose device) used for the intranasal migraine medications Imitrex™ (sumatriptan) and Zomig™ (zolmitriptan) and about half of that for a bi-dose design. Sterile drug products may be produced using aseptic processing or terminal sterilization. Terminal sterilization usually involves filling and sealing product containers under high-quality environmental conditions. Products are filled and sealed in this type of environment to minimize the microbial and particulate content of the in-process product and to help ensure that the subsequent sterilization process is successful. In most cases, the product, container, and closure have low bioburden, but they are not sterile. The product in its final container is then subjected to a sterilization process such as heat or irradiation. In an aseptic process, the drug product, container, and closure are first subjected to sterilization methods separately, as appropriate, and then brought together. Because there is no process to sterilize the product in its final container, it is critical that containers be filled and sealed in an extremely high-quality environment. Aseptic processing involves more variables than terminal sterilization. Before aseptic assembly into a final product, the individual parts of the final product are generally subjected to various sterilization processes. For example, glass containers are subjected to dry heat; rubber closures are subjected to moist heat; and liquid dosage forms are subjected to filtration. Each of these manufacturing processes requires validation and control.

Devices recited herein may employ any of the pharmaceutical formulations, and are useful in all of the methods disclosed herein.

### Pharmaceutical Compositions

Also provided are pharmaceutical compositions comprising the opioid antagonist naltrexone. In some embodiments, the pharmaceutical compositions comprise the opioid antagonist naltrexone and a pharmaceutically acceptable carrier. The carrier(s) must be “acceptable” in the sense of being compatible with the other ingredients of the formulation and not overly deleterious to the recipient thereof. Some embodiments of the present disclosure include a method of producing a pharmaceutical composition comprising admixing the opioid antagonist naltrexone and a pharmaceutically acceptable carrier. Pharmaceutical compositions are applied directly to the nasal cavity using the devices described herein. In the case of a spray, this may be achieved for example by means of a metering atomizing spray pump.

Liquid preparations include solutions, suspensions and emulsions, for example, water or water-propylene glycol solutions. Additional ingredients in liquid preparations may include: antimicrobial preservatives, such as benzalkonium chloride, methylparaben, sodium benzoate, benzoic acid, phenyl ethyl alcohol, and the like, and mixtures thereof; surfactants such as Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and the like, and mixtures thereof; a tonicity agent such as: dextrose, lactose, sodium chloride, calcium chloride, magnesium chloride, sorbitol, sucrose, mannitol, trehalose, raffinose, polyethylene glycol, hydroxyethyl starch, glycine, and the like, and mixtures thereof, and a suspending agent such as microcrystalline cellulose, carboxymethylcellulose sodium NF, polyacrylic acid, magnesium aluminum silicate, xanthan gum, and the like, and mixtures thereof.

In other embodiments, the opioid antagonist is absorbed quickly, i.e., within less than about 10 minutes and/or yielding a time to the maximum plasma concentration (Tₘₐₓ) of about 15 to about 30 minutes. For example, in certain embodiments, the opioid antagonist is absorbed within the first 10 min after administration and the time to the maximum plasma concentration (Tₘₐₓ) is 15 min or less. In some embodiments, the plasma concentration versus time curve of the opioid antagonist in the patient has a Tₘₐₓ of less than 30 minutes. In some embodiments, the plasma concentration versus time curve of the opioid antagonist in the patient has a Tₘₐₓ of less than 25 minutes. In some embodiments, the plasma concentration versus time curve of the opioid antagonist in the patient has a Tₘₐₓ of less than 20 minutes. In some embodiments, the plasma concentration versus time curve of the opioid antagonist in the patient has a Tₘₐₓ of less than 15 minutes. In some embodiments, the plasma concentration versus time curve of the opioid antagonist in the patient has a Tₘₐₓ of less than 10 minutes. In some embodiments, the plasma concentration versus time curve of the opioid antagonist in the patient has a Tm Tₘₐₓ ax of less than 5 minutes. In some embodiments, the plasma concentration versus time curve of the opioid antagonist in the patient has a Tₘₐₓ of about 25 minutes. In some embodiments, the plasma concentration versus time curve of the opioid antagonist in the patient has a Tm Tₘₐₓ ax of about 20 minutes. In some embodiments, the plasma concentration versus time curve of the opioid antagonist in the patient has a Tₘₐₓ of about 15 minutes. In some embodiments, the plasma concentration versus time curve of the opioid antagonist in the patient has a Tₘₐₓ of about 10 minutes. In some embodiments, the plasma concentration versus time curve of the opioid antagonist in the patient has a Tₘₐₓ of about 5 minutes.

In some embodiments, delivery of the therapeutically effective amount to the patient, provides occupancy at Tₘₐₓ of the opioid antagonist at the opioid receptors in the respiratory control center of the patient of greater than about 90%. In some embodiments, delivery of the therapeutically effective amount to the patient, provides occupancy at Tₘₐₓ of the opioid antagonist at the opioid receptors in the respiratory control center of the patient of greater than about 95%. In some embodiments, delivery of the therapeutically effective amount to the patient, provides occupancy at Tₘₐₓ of the opioid antagonist at the opioid receptors in the respiratory control center of the patient of greater than about 99%.

In some embodiments, the relative bioavailability (comparing the dose-adjusted AUC_{0-inf} after IN administration to that of the IM formulation) of naltrexone in a formulation as disclosed herein, will be about 40% to about 80%. In some embodiments, the relative bioavailability will be about 45% to about 75%. In some embodiments, the relative bioavailability will be about 50% to about 70%. In some embodiments, the relative bioavailability will be about 5% to about 65%. In some embodiments, the relative bioavailability will be about 60%.

In some embodiments, the pharmaceutical composition comprises about 1-10 mg naltrexone hydrochloride, or a hydrate thereof, formulated for intranasal administration, and produces a plasma concentration versus time curve having an area under the curve (AUC) that is about 60% of the AUC for 1.5 mg IM naltrexone.

In some embodiments, the patient to be treated is an opioid overdose patient or a suspected opioid overdose patient.

In some embodiments, the patient is in a lying, supine, or recovery position. In some embodiments, the patient is in a lying position. In some other embodiments, the patient is in a supine position. In some embodiments, the patient is in a recovery position.

In some embodiments, the therapeutically effective amount of an opioid antagonist is delivered by an untrained individual.

In some embodiments, the therapeutically effective amount is equivalent to about 1.5 mg of naltrexone hydrochloride. In some embodiments, the therapeutically effective amount is equivalent to about 2 mg of naltrexone hydrochloride. In some embodiments, the therapeutically effective amount is equivalent to about 2.5 mg of naltrexone hydrochloride. In some embodiments, the therapeutically effective amount is equivalent to about 3 mg of naltrexone hydrochloride. In some embodiments, the therapeutically effective amount is equivalent to about 3.5 mg of naltrexone hydrochloride. In some embodiments, the therapeutically effective amount is equivalent to about 4 mg of naltrexone hydrochloride. In some embodiments, the therapeutically effective amount is equivalent to about 4.5 mg of naltrexone hydrochloride. In some embodiments, the therapeutically effective amount is equivalent to about 5 mg of naltrexone hydrochloride. In certain embodiments, the opioid antagonist is the only pharmaceutically active compound in the pharmaceutical composition.

In some embodiments, the pharmaceutical composition comprises a solution of naltrexone hydrochloride, or a hydrate thereof.

In some embodiments, the volume of the pharmaceutical composition in the reservoir is not more than about 140 μL.

In some embodiments, about 100 μL of the pharmaceutical composition in the reservoir is delivered to the patient in one actuation.

In some embodiments, the pharmaceutical composition further comprises one or more excipients selected from water and NaCl.

In some embodiments, the pharmaceutical composition is substantially free of antimicrobial preservatives.

The use of absorption enhancers, such as alkylsaccharides, cyclodextrins, and chitosans, may increase the rate at which naltrexone is absorbed and decrease the Tₘₐₓ. Such absorption enhancers typically operate by affecting two primary mechanisms for nasal absorption: paracellular transport via opening of tight junctions between cells, and transcellular transport or transcytosis through cells via vesicle carriers.

For example, alkylsaccharides are used in commercial food and personal care products and have been designated Generally Recognized as Safe (GRAS) substances for food applications. They are non-irritating enhancers of transmucosal absorption that are odorless, tasteless, non-toxic, non-mutagenic, and non-sensitizing in the Draize test up to a 25% concentration. Alkylsaccharides increase absorption by increasing paracellular permeability, as indicated by a decrease in transepithelial electrical resistance; they may also increase transcytosis. The effect may be short-lived.

When an alkylsaccharide is added to an intranasal formulation, the maximum plasma concentration can increase several-fold in comparison to Imitrex nasal spray, and the Tₘₐₓ can be reduced from hours to minutes. Total exposure, as measured by the area under the concentration-time curve (AUC), can increase by about 30%. This increase in AUC of naltrexone could not be predicted by one of skill in the art, because structurally similar drugs, such as nalmefene, do not have increased AUC under these same conditions. An intranasal formulation of naltrexone has the potential to be used for treating AUD without the use of needles or an extended-release formulation.

Some absorption enhancing excipients can alter the paracellular and/or transcellular pathways, others can extend residence time in the nasal cavity or prevent metabolic changes. Without an absorption enhancer, the molecular-weight limit for nasal absorption is about 1 kDa, while administration of drugs in conjunction with absorption enhancers can enable the absorption of molecules from 1-30 kDa. Intranasal administration of most absorption enhancers, however, can cause nasal mucosa damage. Maggio, J. Excipients and Food Chem. 5(2):100-12, 2014. Examples of absorption enhancers include aprotinin, benzalkonium chloride, benzyl alcohol, capric acid, ceramides, cetylpyridinium chloride, chitosan, cyclodextrins, deoxycholic acid, decanoyl carnitine, EDTA, glycocholic acid, glycodeoxycholic acid, glycofurol, glycosylated sphingosines, glycyrrhetinic acids, 2-hydroxypropyl-β-cyclodextrin, laureth-9, lauric acid, lauroyl carnitine, lauryl sulfate, lysophosphatidylcholine, menthol, poloxamer 407, poloxamer F68, poly-L-arginine, polyoxyethylene-9-lauryl ether, polysorbate 80, propylene glycol, quillaia saponin, salicylic acid, β-sitosterol-β-D-glucoside, sucrose cocoate, taurocholic acid, taurodeoxycholic acid, taurodihydrofusidic acid, and alkylsaccharides, such as dodecyl maltoside, tetradecyl maltoside and sucrose dodecanoate.

The opioid antagonist naltrexone described herein can be formulated into pharmaceutical compositions using techniques well known to those in the art. Suitable pharmaceutically acceptable carriers, outside those mentioned herein, are known in the art.

The opioid antagonist naltrexone described herein may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like. The acid addition salts may be obtained as the direct products of compound synthesis. In the alternative, the free base may be dissolved in a suitable solvent containing the appropriate acid and the salt isolated by evaporating the solvent or otherwise separating the salt and solvent. The opioid antagonist naltrexone described herein may form solvates with standard low molecular weight solvents using methods known to the skilled artisan.

Accordingly, provided herein are pharmaceutical formulations for intranasal administration comprising naltrexone. In certain embodiments, the formulation is an aqueous solution. In certain embodiments, the formulation comprises, per dose, between about 25 and about 200 μL of the aqueous solution. In certain embodiments, the formulation comprises, per dose, between about 50 and about 200 μL of the aqueous solution. In certain embodiments, the formulation comprises, per dose, not more than about 140 μL. In certain embodiments, the formulation comprises, per dose, not more than about 100 μL. The formulation may comprise, per dose, about 25 μL, about 50 μL, about 75 μL, about 100 μL, about 125 μL, about 150 μL, about 175 μL, or about 200 μL of the aqueous solution.

In certain embodiments, the formulation comprises between about 1% (w/v) and about 16% (w/v) of the opioid antagonist naltrexone. In certain embodiments, the formulation comprises between about 2% (w/v) and about 12% (w/v) of naltrexone. In certain embodiments, the formulation comprises between about 2% (w/v) and about 10% (w/v) of naltrexone. In certain embodiments, the formulation comprises between about 2% (w/v) and about 8% (w/v) of naltrexone. In certain embodiments, the formulation comprises between about 2% (w/v) and about 4% (w/v) of naltrexone. In certain embodiments, the formulation comprises about 1% (w/v), about 2% (w/v), about 3% (w/v), about 4% (w/v), about 5% (w/v), about 6% (w/v), about 7% (w/v), or about 8% (w/v) of naltrexone. In certain embodiments, the formulation comprises about 1% (w/v) of naltrexone. In certain embodiments, the formulation comprises about 2% (w/v) of naltrexone. In certain embodiments, the formulation comprises about 4% (w/v) of naltrexone.

In certain embodiments, the formulation comprises between about 1 mg and about 16 mg of the opioid antagonist naltrexone. In certain embodiments, the formulation comprises between about 2 mg and about 12 mg of naltrexone. In certain embodiments, the formulation comprises between about 2 mg and about 10 mg of naltrexone. In certain embodiments, the formulation comprises between about 2 mg and about 8 mg of naltrexone. In certain embodiments, the formulation comprises between about 2 mg and about 4 mg of naltrexone. In certain embodiments, the formulation comprises about 1 mg, about 2 mg, about 3 mg, about 4 mg, about 5 mg, about 6 mg, about 7 mg, or about 8 mg of naltrexone. In certain embodiments, the formulation comprises about 1 mg of naltrexone. In certain embodiments, the formulation comprises about 2 mg of naltrexone. In certain embodiments, the formulation comprises about 4 mg of naltrexone.

In certain embodiments, provided herein are pharmaceutical formulations for intranasal administration comprising, in an aqueous solution of not more than about 140 μL:

between about 2 mg and about 16 mg of naltrexone; and

between about 0.2 mg and about 1.2 mg of an isotonicity agent.
In certain embodiments, provided herein are pharmaceutical formulations for intranasal administration comprising, in an aqueous solution of not more than about 140 μL:

between about 2% (w/v) and about 16% (w/v) of naltrexone; and

between about 0.2% (w/v) and about 1.2% (w/v) of an isotonicity agent.

In certain embodiments, the pharmaceutical formulation comprises:

about 2 mg or about 4 mg naltrexone hydrochloride or a hydrate thereof; and

between about 0.2 mg and about 1.2 mg of an isotonicity agent.

In certain embodiments, the pharmaceutical formulation comprises:

about 2% (w/v) or about 4% (w/v) naltrexone hydrochloride or a hydrate thereof; and

between about 0.2% (w/v) and about 1.2% (w/v) of an isotonicity agent.

In certain embodiments, the isotonicity agent is sodium chloride.

In certain embodiments, the pharmaceutical formulation comprises:

about 2 mg or about 4 mg naltrexone hydrochloride; and

about 0.74 mg sodium chloride.

In certain embodiments, the pharmaceutical formulation comprises:

about 4 mg naltrexone hydrochloride; and

about 0.74 mg sodium chloride.

In certain embodiments, provided herein are pharmaceutical formulations above comprise an aqueous solution of not more than about 100 μL.

In certain embodiments, the pharmaceutical formulation comprises about 4 mg or about 4% (w/v) naltrexone hydrochloride or a hydrate thereof. In certain embodiments, the pharmaceutical formulation comprises about 2 mg or about 2% (w/v) naltrexone hydrochloride or a hydrate thereof. In certain embodiments, the naltrexone hydrochloride is provided as naltrexone hydrochloride dihydrate.

In certain embodiments, the pharmaceutical formulation additionally comprises an absorption enhancer. In certain embodiments, the pharmaceutical formulation comprises between about 0.005% to about 2.5% of the absorption enhancer. In certain embodiments, the pharmaceutical formulation comprises between about 0.05% to about 2.5% of the absorption enhancer. In certain embodiments, the pharmaceutical formulation comprises between about 0.1% to about 0.5% of the absorption enhancer. In certain embodiments, the pharmaceutical formulation comprises about 0.25% of the absorption enhancer. In certain embodiments, the pharmaceutical formulation comprises about 0.18% of the absorption enhancer. In certain embodiments, the absorption enhancer is an alkylsaccharide. In certain embodiments, the alkylsaccharide is chosen from dodecyl maltoside, tetradecyl maltoside (TDM) and sucrose dodecanoate.

In certain embodiments, the alkylsaccharide is Intravail® (dodecyl maltoside). Intravail® is the alkyl saccharide 1-O-n-dodecyl-β-D-maltopyranoside (alternately referred to as lauryl-β-D-maltopyranoside, dodecyl maltopyranoside, and DDM; C₂₄H₄₆Q₁₁). Alkylsaccharides are used in commercial food and personal care products and have been designated Generally Recognized as Safe (GRAS) substances for food applications. They are non-irritating enhancers of transmucosal absorption that are odorless, tasteless, non-toxic, non-mutagenic, and non-sensitizing in the Draize test up to a 25% concentration. Alkylsaccharides increase absorption by increasing paracellular permeability, as indicated by a decrease in transepithelial electrical resistance; they may also increase transcytosis. The effect is short-lived. Other alkylsaccharides include tetradecyl maltoside (TDM) and sucrose dodecanoate.

In certain embodiments, the pharmaceutical formulation comprises between about 0.005% to about 0.05% (w/v) of the absorption enhancer. In certain embodiments, the pharmaceutical formulation comprises between about 0.005% to about 0.015% (w/v) of the absorption enhancer. In certain embodiments, the pharmaceutical formulation comprises about 0.01% (w/v) of the absorption enhancer. In certain embodiments, the absorption enhancer is benzalkonium chloride.

In certain embodiments, an intranasal formulation comprises between about 0.05% and about 2.5% (w/v) Intravail®. In certain embodiments, an intranasal formulation comprises between about 0.1% and about 0.5% (w/v) Intravail®. In certain embodiments, an intranasal formulation comprises between about 0.15% and about 0.35% (w/v) Intravail®. In certain embodiments, an intranasal formulation comprises between about 0.15% and about 0.2% (w/v) Intravail®. In certain embodiments, an intranasal formulation comprises about 0.18% (w/v) Intravail®. In certain embodiments, an intranasal formulation comprises about 0.2% to about 0.3% (w/v) Intravail®. In certain embodiments, an intranasal formulation comprises about 0.25% (w/v) Intravail®.

When 0.18% Intravail® was added to an intranasal formulation of sumatriptan, the maximum plasma concentration increased almost four-fold in comparison to Imitrex nasal spray and Tₘₐₓ was reduced from 1-2 hours to 8-10 minutes. Total exposure, as measured by the area under the concentration-time curve (AUC), increased 32%. An intranasal formulation of naltrexone has the potential to be used for treating AUD without the use of needles or an extended-release formulation. Inclusion of Intravail® may improve pharmacokinetic parameters in some applications.

In certain embodiments, the pharmaceutical formulation additionally comprises an isotonicity agent. The intranasal formulation may comprise between about 0.2% (w/v) and about 1.2% (w/v) isotonicity agent, such as about 0.2% (w/v), about 0.3% (w/v), about 0.4% (w/v), about 0.5% (w/v), about 0.6% (w/v), about 0.7% (w/v), about 0.8% (w/v), about 0.9% (w/v), about 1.0% (w/v), about 1.1% (w/v), or about 1.2% (w/v). The intranasal formulation may comprise more than about 0.1% (w/v) isotonicity agent. The intranasal formulation may comprise less than about 1.2% (w/v) isotonicity agent.

In certain embodiments, provided herein are pharmaceutical formulations for intranasal administration comprising, in an aqueous solution of not more than about 140 μL:
between about 2 mg and about 16 mg of naltrexone;
about 0.005 mg to about 2.5 mg of an absorption enhancer; and
between about 0.2 mg and about 1.2 mg of an isotonicity agent.
In certain embodiments, provided herein are pharmaceutical formulations for intranasal administration comprising, in an aqueous solution of not more than about 140 μL:
between about 2% (w/v) and about 16% (w/v) of naltrexone;
about 0.005% (w/v) to about 2.5% (w/v) of an absorption enhancer; and
between about 0.2% (w/v) and about 1.2% (w/v) of an isotonicity agent.
In certain embodiments, the pharmaceutical formulation comprises:
about 2 mg or about 4 mg naltrexone hydrochloride or a hydrate thereof;
about 0.005 mg to about 2.5 mg of an absorption enhancer; and
between about 0.2 mg and about 1.2 mg of an isotonicity agent.
In certain embodiments, the pharmaceutical formulation comprises:
about 2% (w/v) or about 4% (w/v) naltrexone hydrochloride or a hydrate thereof;
about 0.005% (w/v) to about 2.5% (w/v) of an absorption enhancer; and
between about 0.2% (w/v) and about 1.2% (w/v) of an isotonicity agent.

In certain embodiments, provided herein are pharmaceutical formulations for intranasal administration comprising, in an aqueous solution of not more than about 140 μL:
between about 2 mg and about 16 mg of naltrexone;
about 0.05 mg to about 2.5 mg of an absorption enhancer; and
between about 0.2 mg and about 1.2 mg of an isotonicity agent.
In certain embodiments, provided herein are pharmaceutical formulations for intranasal administration comprising, in an aqueous solution of not more than about 140 μL:
between about 2% (w/v) and about 16% (w/v) of naltrexone;
about 0.05% (w/v) to about 2.5% (w/v) of an absorption enhancer; and
between about 0.2% (w/v) and about 1.2% (w/v) of an isotonicity agent.
In certain embodiments, the pharmaceutical formulation comprises:
about 2 mg or about 4 mg naltrexone hydrochloride or a hydrate thereof;
about 0.05 mg to about 2.5 mg of an absorption enhancer; and
between about 0.2 mg and about 1.2 mg of an isotonicity agent.
In certain embodiments, the pharmaceutical formulation comprises:
about 2% (w/v) or about 4% (w/v) naltrexone hydrochloride or a hydrate thereof;
about 0.05% (w/v) to about 2.5% (w/v) of an absorption enhancer; and
between about 0.2% (w/v) and about 1.2% (w/v) of an isotonicity agent.

In certain embodiments, provided herein are pharmaceutical formulations above comprise an aqueous solution of not more than about 100 μL.

In certain embodiments, provided herein are pharmaceutical formulations for intranasal administration comprising, in an aqueous solution of not more than about 140 μL:
between about 2 mg and about 16 mg of naltrexone;
about 0.005 mg to about 0.015 mg of an absorption enhancer; and
between about 0.2 mg and about 1.2 mg of an isotonicity agent.

In certain embodiments, provided herein are pharmaceutical formulations for intranasal administration comprising, in an aqueous solution of not more than about 140 μL:
between about 2% (w/v) and about 16% (w/v) of naltrexone;
about 0.005% (w/v) to about 0.015% (w/v) of an absorption enhancer; and
between about 0.2% (w/v) and about 1.2% (w/v) of an isotonicity agent.

In certain embodiments, the pharmaceutical formulation comprises:
about 2 mg or about 4 mg naltrexone hydrochloride or a hydrate thereof;
about 0.005 mg to about 0.015 mg of an absorption enhancer; and
between about 0.2 mg and about 1.2 mg of an isotonicity agent.

In certain embodiments, the pharmaceutical formulation comprises:
about 2% (w/v) or about 4% (w/v) naltrexone hydrochloride or a hydrate thereof;
about 0.005% (w/v) to about 0.015% (w/v) of an absorption enhancer; and
between about 0.2% (w/v) and about 1.2% (w/v) of an isotonicity agent.

In certain embodiments, provided herein are pharmaceutical formulations above comprise an aqueous solution of not more than about 100 μL.

In certain embodiments, the isotonicity agent is sodium chloride.

In certain embodiments, the absorption enhancer is Intravail® (dodecyl maltoside).

In certain embodiments, the pharmaceutical formulation comprises:
about 2 mg or about 4 mg naltrexone hydrochloride;
about 0.25 mg Intravail® (dodecyl maltoside); and
about 0.74 mg sodium chloride.

In certain embodiments, the pharmaceutical formulation comprises:
about 4 mg naltrexone hydrochloride;
about 0.25 mg Intravail® (dodecyl maltoside); and
about 0.74 mg sodium chloride.

In certain embodiments, the absorption enhancer is benzalkonium chloride.

In certain embodiments, the pharmaceutical formulation comprises:
about 2 mg or about 4 mg naltrexone hydrochloride;
about 0.01 mg benzalkonium chloride; and
about 0.74 mg sodium chloride.

In certain embodiments, the pharmaceutical formulation comprises:
about 4 mg naltrexone hydrochloride;
about 0.01 mg benzalkonium chloride; and
about 0.74 mg sodium chloride.

In certain embodiments, provided herein are pharmaceutical formulations above comprise an aqueous solution of not more than about 100 μL.

In certain embodiments, the pharmaceutical formulation comprises about 4 mg or about 4% (w/v) naltrexone hydrochloride or a hydrate thereof. In certain embodiments, the pharmaceutical formulation comprises about 2 mg or about 2% (w/v) naltrexone hydrochloride or a hydrate thereof. In certain embodiments, the naltrexone hydrochloride is provided as naltrexone hydrochloride dihydrate.

In certain embodiments, the pharmaceutical formulation additionally comprises a compound which is a preservative and/or surfactant.

In certain embodiments, the preservative and/or surfactant is chosen from benzalkonium chloride, methylparaben, sodium benzoate, benzoic acid, phenyl ethyl alcohol, and the like, and mixtures thereof surfactants such as Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and the like, and mixtures thereof.

In certain embodiments, the pharmaceutical formulation additionally comprises a stabilizing agent.

In certain embodiments, the stabilizing agent is disodium edetate (EDTA).

In some embodiments the acid or base, is sufficient to achieve a pH of about 3.5-4.0. In some embodiments the acid or base, is sufficient to achieve a pH of about 3.5-4.5. In some embodiments the acid or base, is sufficient to achieve a pH of about 4.0-4.5. In some embodiments the acid or base, is sufficient to achieve a pH of about 3, about 3.5, about 4, about 4.5, about 5, about 5.5, about 6, or about 7.

In some embodiments, the preservative, absorption enhancer and/or a cationic surfactant is selected from benzalkonium chloride, cyclodextrins, an alkylsaccharide (e.g., a nonionic alkylsaccharide surfactant such as an alkylglycoside and a sucrose ester of fatty acids that consists of an aliphatic hydrocarbon chain coupled to a sugar moiety by a glycosidic or ester bond, respectively), fusidic acid derivatives, phosphatidylcholines, microspheres and liposomes, and bile salts. In a particular embodiment, the preservative, absorption enhancer and/or a cationic surfactant is benzalkonium chloride.

provided herein are pharmaceutical composition further comprises one or more excipients selected from water, NaCl, benzalkonium chloride, sodium edetate, disodium edetate, and hydrochloric acid. In some embodiments, the pharmaceutical composition further comprises water, NaCl, benzalkonium chloride, disodium edetate, and hydrochloric acid.

In some embodiments, the pharmaceutical composition comprises benzalkonium chloride. The benzalkonium chloride can function as a preservative (even in low amounts), an absorption enhancer, and/or a cationic surfactant (typically at a higher amount formulations for these latter two). Benzalkonium chloride is represented by the following structure:

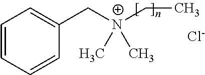

in which n is an integer, and a mixture of more than one thereof can be used. In some embodiments, n is 8, 10, 12, 14, 16, or 18, and in some embodiments, n is 10, 12, or 14. In some embodiments, the pharmaceutical composition comprises about 0.005% to about 1% benzalkonium chloride.

Also provided herein are pharmaceutical formulations for intranasal administration comprising, in an aqueous solution of not more than about 200 μL:
between about 2 mg and about 16 mg of naltrexone;
between about 0.2 mg and about 1.2 mg of an isotonicity agent;
optionally, between about 0.005 mg and about 0.015 mg of a compound which is a preservative and/or cationic surfactant;
optionally, between about 0.005% to about 2.5% of an absorption enhancer;
optionally, between about 0.1 mg and about 0.5 mg of a stabilizing agent; and
an amount of an acid sufficient to achieve a pH of 3.5-5.5.

In certain embodiments, the pharmaceutical formulation comprises:
between about 2 mg and about 16 mg of naltrexone;
between about 0.2 mg and about 1.2 mg of an isotonicity agent;
between about 0.005 mg and about 0.015 mg of a compound which is a preservative and/or cationic surfactant;
between about 0.005 and about 0.70 mg of a compound which is an absorption enhancer;
between about 0.1 mg and about 0.5 mg of a stabilizing agent; and
an amount of an acid sufficient to achieve a pH of 3.5-5.5.

In certain embodiments, the pharmaceutical formulation comprises:
about 2 mg or about 4 mg naltrexone hydrochloride or a hydrate thereof;
between about 0.2 mg and about 1.2 mg of an isotonicity agent;
between about 0.005 mg and about 0.015 mg of a compound which is a preservative and/or cationic surfactant;
between about 0.005 and about 0.70 mg of a compound which is an absorption enhancer;
between about 0.1 mg and about 0.5 mg of a stabilizing agent;
an amount of hydrochloric acid sufficient to achieve a pH of 3.5-5.5.

In certain embodiments, the isotonicity agent is sodium chloride. In certain embodiments, the preservative and/or cationic surfactant is benzalkonium chloride. In certain embodiments, the absorption enhancer is chosen from benzalkonium chloride, chitosan, cyclodextrins, deoxycholic acid, dodecyl maltoside, glycocholic acid, laureth-9, taurocholic acid, and taurodihydrofusidic acid. In certain embodiments, the absorption enhancer is Intravail®. In certain embodiments, the stabilizing agent is edetate disodium. In certain embodiments, the acid is hydrochloric acid.

In certain embodiments, the pharmaceutical formulation comprises:
about 2 mg or about 4 mg naltrexone hydrochloride or a hydrate thereof;
about 0.74 mg sodium chloride;
about 0.01 mg benzalkonium chloride;
about 0.25 mg Intravail® (dodecyl maltoside);
about 0.2 mg edetate disodium; and
an amount of hydrochloric acid sufficient to achieve a pH of 3.5-5.5.

In certain embodiments, the pharmaceutical formulation comprises:
about 2 mg or about 4 mg naltrexone hydrochloride or a hydrate thereof;
about 0.74 mg sodium chloride;
about 0.01 mg benzalkonium chloride;
about 0.2 mg edetate disodium; and
an amount of hydrochloric acid sufficient to achieve a pH of 3.5-5.5.

In certain embodiments, the pharmaceutical formulation comprises:
about 2 mg or about 4 mg naltrexone hydrochloride or a hydrate thereof;
about 0.74 mg sodium chloride;
about 0.25 mg Intravail® (dodecyl maltoside);
about 0.2 mg edetate disodium; and
an amount of hydrochloric acid sufficient to achieve a pH of 3.5-5.5.

In certain embodiments, the pharmaceutical formulation comprises about 4 mg naltrexone hydrochloride or a hydrate thereof. In certain embodiments, the pharmaceutical formulation comprises between about 2.5 mg and about 8 mg naltrexone hydrochloride or a hydrate thereof. In certain embodiments, the pharmaceutical formulation comprises about 2 mg naltrexone hydrochloride or a hydrate thereof. In certain embodiments, the pharmaceutical formulation comprises about 2.5 mg naltrexone hydrochloride or a hydrate thereof. In certain embodiments, the pharmaceutical formulation comprises about 4 mg naltrexone hydrochloride dihydrate.

Also provided herein are pharmaceutical formulations for intranasal administration comprising, in an aqueous solution of about 100 μL:

about 4 mg naltrexone hydrochloride or a hydrate thereof;

between about 0.2 mg and about 1.2 mg of an isotonicity agent;
between about 0.005 mg and about 0.015 mg of a compound which is a preservative and/or cationic surfactant;
between about 0.00 and about 0.50 mg of a compound which is an absorption enhancer;

between about 0.1 mg and about 0.5 mg of a stabilizing agent; and

an amount of an acid sufficient to achieve a pH of 3.5-5.5.

Also provided herein are pharmaceutical formulations for intranasal administration comprising, in an aqueous solution of about 100 μL:

about 4 mg naltrexone hydrochloride or a hydrate thereof;

between about 0.2 mg and about 1.2 mg of an isotonicity agent;
optionally, between about 0.005 mg and about 0.015 mg of a compound which is a preservative and/or cationic surfactant;
between about 0.005 and about 0.50 mg of a compound which is an absorption enhancer;

optionally, between about 0.1 mg and about 0.5 mg of a stabilizing agent; and

an amount of an acid sufficient to achieve a pH of 3.5-5.5.

Also provided herein are pharmaceutical formulations for intranasal administration comprising, in an aqueous solution of about 100 μL:

about 4 mg naltrexone hydrochloride or a hydrate thereof;

between about 0.2 mg and about 1.2 mg of an isotonicity agent;
between about 0.005 mg and about 0.015 mg of a compound which is a preservative and/or cationic surfactant;

between about 0.05 and about 0.50 mg of a compound which is an absorption enhancer;

between about 0.1 mg and about 0.5 mg of a stabilizing agent; and

an amount of an acid sufficient to achieve a pH of 3.5-5.5.

In certain embodiments, the pharmaceutical formulation comprises:

about 4 mg naltrexone hydrochloride or a hydrate thereof;

about 0.74 mg sodium chloride;

about 0.01 mg benzalkonium chloride;

about 0.18 mg Intravail® (dodecyl maltoside);

about 0.2 mg edetate disodium; and

an amount of hydrochloric acid sufficient to achieve a pH of 3.5-5.5.

Also provided herein are pharmaceutical formulations for intranasal administration comprising, in an aqueous solution of about 100 μL:

about 2 mg naltrexone hydrochloride or a hydrate thereof;

between about 0.2 mg and about 1.2 mg of an isotonicity agent;

between about 0.005 mg and about 0.015 mg of a compound which is a preservative and/or cationic surfactant;
between about 0.00 and about 0.50 mg of a compound which is an absorption enhancer;

between about 0.1 mg and about 0.5 mg of a stabilizing agent; and

an amount of an acid sufficient to achieve a pH of 3.5-5.5.

In certain embodiments, the pharmaceutical formulation comprises:

about 2 mg naltrexone hydrochloride dihydrate;

about 0.74 mg sodium chloride;

about 0.01 mg benzalkonium chloride;

about 0.18 mg Intravail® (dodecyl maltoside);

about 0.2 mg edetate disodium; and

an amount of hydrochloric acid sufficient to achieve a pH of 3.5-5.5.

In certain embodiments, the therapeutically effective amount comprises about 2 to about 16 mg of naltrexone. In certain embodiments, the pharmaceutical formulation comprises an amount equivalent to about 2, about 3, about 4, about 5, about 6, about 7, about 8, about 9, about 10, about 11, about 12, about 13, about 14, about 15, or about 16 mg of naltrexone hydrochloride. In certain embodiments, the pharmaceutical formulation comprises an amount equivalent to about 4 mg to about 8 mg of naloxone hydrochloride. In certain embodiments, the pharmaceutical formulation comprises an amount equivalent to about 16 mg of naloxone hydrochloride.

In certain embodiments, the pharmaceutical composition is in an aqueous solution of about 100 μL.

In certain embodiments, upon nasal delivery of said pharmaceutical composition to said patient, less than about 10% of said pharmaceutical composition leaves the nasal cavity via drainage into the nasopharynx or externally.

### Indications

Also provided are pharmaceutical compositions in a device adapted for nasal delivery to a subject at risk for opioid overdose, comprising a therapeutically effective amount of the opioid antagonist naltrexone and pharmaceutically acceptable salts thereof. In some embodiments, the device is pre-primed. In some embodiments, the device can be primed before use. In some embodiments, the device can be actuated with one hand.

In some embodiments, the opioid antagonist is for use in the treatment of an opioid overdose symptom. In some embodiments, the opioid overdose symptom is selected from: respiratory depression, altered level consciousness, miotic pupils, cardiovascular depression, hypoxemia, acute lung injury, aspiration pneumonia, sedation, and hypotension. In some embodiments, the opioid antagonist is for use in the emergency treatment of known or suspected opioid overdose, as manifested by one or more symptoms selected from: respiratory depression and central nervous system depression. In some embodiments, the opioid antagonist is for use in the emergency treatment of known or suspected opioid overdose characterized by one or more symptoms selected from: decreased breathing rate, decreased heart rate, and loss of consciousness. In some embodiments, the symptom is respiratory depression. In some embodiments, the opioid antagonist is for use in the complete or partial reversal of narcotic depression, including respiratory depression, induced by opioids selected from: natural and synthetic narcotics, propoxyphene, methadone, nalbuphine, pentazocine and butorphanol.

In some embodiments, the opioid overdose symptom is respiratory depression induced by opioids. In some embodiments, the respiratory depression is induced by opioids selected from: natural and synthetic narcotics, propoxyphene, methadone, nalbuphine, pentazocine and butorphanol. In some embodiments, the respiratory depression is induced by an opioid agonist selected from codeine, morphine, methadone, fentanyl, oxycodone HCl, hydrocodone bitartrate, hydromorphone, oxymorphone, meperidine, propoxyphene, opium, heroin, tramadol, and tapentadol. In some embodiments, the respiratory depression is caused by the illicit use of opioids or by an accidental misuse of opioids during medical opioid therapy. In some embodiments, the symptom is caused by misuse of the opioid agonist.

In some embodiments, the patient is in a lying position. In some embodiments, the patient is in a supine position. In some embodiments, the patient is in a prone position. In some embodiments, the patient is not breathing. In some embodiments, the patient is an opioid overdose patient. In some embodiments, the therapeutically effective amount of an opioid antagonist is delivered by an untrained individual.

In some embodiments, the patient is free from respiratory depression for at least about 1 hour following treatment consisting essentially of delivery of the therapeutically effective amount of the opioid antagonist. In some embodiments, the patient is free from respiratory depression for at least about 2 hours following treatment consisting essentially of delivery of the therapeutically effective amount of the opioid antagonist. In some embodiments, the patient is free from respiratory depression for at least about 3 hours following treatment consisting essentially of delivery of the therapeutically effective amount of the opioid antagonist. In some embodiments, the patient is free from respiratory depression for at least about 4 hours following treatment consisting essentially of delivery of the therapeutically effective amount of the opioid antagonist. In some embodiments, the patient is free from respiratory depression for at least about 5 hours following treatment consisting essentially of delivery of the therapeutically effective amount of the opioid antagonist. In some embodiments, the patient is free from respiratory depression for at least about 6 hours following treatment consisting essentially of delivery of the therapeutically effective amount of the opioid antagonist.

In some embodiments, the patient is free from respiratory depression for at least about 8 hours following treatment comprising delivery of the therapeutically effective amount of the opioid antagonist. In some embodiments, the patient is free from respiratory depression for at least about 10 hours following treatment comprising delivery of the therapeutically effective amount of the opioid antagonist. In some embodiments, the patient is free from respiratory depression for at least about 12 hours following treatment comprising delivery of the therapeutically effective amount of the opioid antagonist. In some embodiments, the patient is free from respiratory depression for at least about 14 hours following treatment comprising delivery of the therapeutically effective amount of the opioid antagonist. In some embodiments, the patient is free from respiratory depression for at least about 16 hours following treatment comprising delivery of the therapeutically effective amount of the opioid antagonist. In some embodiments, the patient is free from respiratory depression for at least about 1 hour to at least about 15 hours following treatment comprising delivery of the therapeutically effective amount of the opioid antagonist. In some embodiments, the patient is free from respiratory depression for at least about 3 hours to at least about 15 hours following treatment comprising delivery of the therapeutically effective amount of the opioid antagonist. In some embodiments, the patient is free from respiratory depression for at least about 3 hours to at least about 12 hours following treatment comprising delivery of the therapeutically effective amount of the opioid antagonist. In some embodiments, the patient is free from respiratory depression for at least about 3 hours to at least about 10 hours following treatment comprising delivery of the therapeutically effective amount of the opioid antagonist. In some embodiments, the patient is free from respiratory depression for at least about 3 hours to at least about 8 hours following treatment comprising delivery of the therapeutically effective amount of the opioid antagonist.

Also provided herein is a single-use, pre-primed device adapted for nasal delivery of a pharmaceutical composition to a patient by one actuation of the device into one nostril of the patient, having a single reservoir comprising about 100 μL of a pharmaceutical composition which is an aqueous solution comprising: about 1 mg or about 10 mg naltrexone hydrochloride or a hydrate thereof between about 0.2 mg and about 1.2 mg of an isotonicity agent; between about 0.005 mg and about 2.5 mg of a compound which acts as a preservative, cationic surfactant, and/or absorption enhancer; between about 0.1 mg and about 0.5 mg of a stabilizing agent; an amount of an acid or base sufficient to achieve a pH of 3.5-5.5.

In some embodiments, the device comprises any of the amounts of naltrexone hydrochloride provided above, for example, between about 1 mg and about 10 mg of the naltrexone hydrochloride or a hydrate thereof. In some embodiments, the device comprises about 3 mg naltrexone hydrochloride or a hydrate thereof.

In some embodiments, the isotonicity agent is NaCl; the compound which is a preservative, cationic surfactant, and/or absorption enhancer is benzalkonium chloride; the stabilizing agent is disodium edetate; and the acid is hydrochloric acid or the base is sodium hydroxide.

In some embodiments, the device comprises: about 1 mg or about 10 mg naltrexone hydrochloride; about 0.74 mg NaCl; about 0.01 mg benzalkonium chloride; about 0.2 mg disodium edetate; and an amount of hydrochloric acid or sodium hydroxide sufficient to achieve a pH of 3.5-5.5.

Also provided are pharmaceutical compositions in a device adapted for nasal delivery to a subject suffering from an opioid-receptor-mediated disease, disorder, or condition, comprising a therapeutically effective amount of the opioid antagonist naltrexone and pharmaceutically acceptable salts thereof. In some embodiments, the device is pre-primed. In some embodiments, the device can be primed before use. In some embodiments, the device can be actuated with one hand.

In some embodiments, the method of treatment employs pharmacological extinction—the use of an opioid antagonist, such as naltrexone, to turn the habit-forming reward-based behavior into a habit-erasing behavior. The effect returns a person's craving for reward to its pre-addiction state.

The method consists of taking an oral dose of naltrexone about 1, about 2, about 3, or about 4 hours before a subject engages in a reward-based behavior. This pre-ingestion dose of oral naltrexone disrupts the body's behavior and reward cycle thereby causing the person to want to do less of the behavior instead of more. Most significantly, studies have shown that this methodology is equally effective with or without therapy, so subjects can choose whether to combine this treatment method with other therapies without negatively impacting the actual physical results. In some embodiments, the method calls for the use of intranasal naltrexone while the individual continues their normal behavior. As a result, maintenance of the medication treatment protocol is expected to be much higher than abstinence alone.

Using this method, extinction of a reward-based disorder can occur within 6 months. However, the efficacy of oral naltrexone is hampered by slow onset, very low bioavailability and high levels of the peripherally selective active metabolite 6-β-naltrexol, and the injectable form of naltrexone presents itself with the obvious difficulties associated with needles including, for example, the need for administration by a practitioner at regularly scheduled intervals. Thus, intranasal administration of naltrexone, and use of absorption enhancers, in a single or multi-use nasal spray pump should significantly improve results in the treatment of reward-based disorders. An intranasal formulation of naltrexone absorbs quickly, providing fast onset of action and high bioavailability without the use of needles.

In some embodiments, upon nasal delivery of the pharmaceutical composition to the patient, less than about 20%, less than about 15%, less than about 10%, or less than about 5%, of the pharmaceutical composition leaves the nasal cavity via drainage into the nasopharynx or externally, as provided above.

In some embodiments, the device is actuatable with one hand.

In some embodiments, the delivery time is less than about 30 seconds. In some embodiments, the delivery time is less than about 25 seconds. In some embodiments, the delivery time is less than about 20 seconds. In some embodiments, the delivery time is less than about 15 seconds.

In some embodiments, the 90% confidence interval for dose delivered per actuation is ±about 2%. In some embodiments, the 95% confidence interval for dose delivered per actuation is ±about 2.5%.

In some embodiments, upon nasal delivery of the pharmaceutical composition to the patient, less than about 20%, less than about 15%, less than about 10%, or less than about 5%, of the pharmaceutical composition leaves the nasal cavity via drainage into the nasopharynx or externally, as provided above.

In some embodiments, the plasma concentration versus time curve of the opioid antagonist in the patient has a Tₘₐₓ of less than 30 minutes, as provided above. In some embodiments, the plasma concentration versus time curve of the opioid antagonist in the patient has a Tₘₐₓ of about 30 minutes. In some embodiments, the plasma concentration versus time curve of the opioid antagonist in the patient has a Tₘₐₓ of about 25 minutes. In some embodiments, the plasma concentration versus time curve of the opioid antagonist in the patient has a Tₘₐₓ of about 20 minutes. In some embodiments, the plasma concentration versus time curve of the opioid antagonist in the patient has a Tₘₐₓ of about 15 minutes. In some embodiments, the plasma concentration versus time curve of the opioid antagonist in the patient has a Tₘₐₓ of about 10 minutes. In some embodiments, the plasma concentration versus time curve of the opioid antagonist in the patient has a Tₘₐₓ of about 5 minutes.

In some embodiments, the patient is free from respiratory depression for at least about 1 hour to at least about 8 hours following treatment comprising delivery of the therapeutically effective amount of the opioid antagonist, as provided above. In some embodiments, the patient is free from respiratory depression for at least about 3 hours to at least about 8 hours following treatment comprising delivery of the therapeutically effective amount of the opioid antagonist.

In some embodiments, said device is filled with said pharmaceutical composition using sterile filling.

In some embodiments, said pharmaceutical composition is storage-stable for about twelve months at about 25° C. and about 60% relative humidity.

In some embodiments, said opioid antagonist is the only pharmaceutically active compound in said pharmaceutical composition.

Also provided are devices as recited in any of the preceding embodiments for use in the treatment of an opioid overdose symptom selected from: respiratory depression, postoperative opioid respiratory depression, altered level consciousness, miotic pupils, cardiovascular depression, hypoxemia, acute lung injury, aspiration pneumonia, sedation, and hypotension.

Also provided are devices as recited in any of the preceding embodiments for use in the reversal of respiratory depression induced by opioids.

In some embodiments, said respiratory depression is caused by the illicit use of opioids or by an accidental misuse of opioids during medical opioid therapy.

Also provided are formulations and devices as recited in any of the preceding embodiments for use in the complete or partial reversal of narcotic depression, including respiratory depression, induced by opioids selected from: natural and synthetic narcotics, propoxyphene, methadone, nalbuphine, pentazocine and butorphanol.

In some embodiments, said patient is an opioid overdose patient or a suspected opioid overdose patient.

In some embodiments, said patient is in a lying, supine, or recovery position. In some embodiments, said patient is in a lying position. In some embodiments, said patient is in a supine position. In some embodiments, said patient is in a recovery position.

In some embodiments, said therapeutically effective amount of an opioid antagonist is delivered by an untrained individual.

In some embodiments, said device is a bi-dose device, wherein a first volume of said pharmaceutical composition is present in a first reservoir and a second volume of said pharmaceutical composition is present in a second reservoir, and wherein said therapeutically effective amount is delivered essentially by a first actuation of said device into a first nostril of said patient and a second actuation of said device into a second nostril of said patient.

In some embodiments, said first volume and said second volume combined is equal to not more than about 380 μL.

In some embodiments, about 100 μL of said first volume of said pharmaceutical composition is delivered by said first actuation.

In some embodiments, about 100 μL of said second volume of said pharmaceutical composition is delivered by said second actuation.

In some embodiments, said bi-dose device is actuatable with one hand.

In some embodiments, the delivery time is less than about 30 seconds. In some embodiments, the delivery time is less than about 25 seconds. In some embodiments, the delivery time is less than about 20 seconds. In some embodiments, the delivery time is less than about 15 seconds.

In some embodiments, the 90% confidence interval for dose delivered per actuation is ±about 2%. In some embodiments, the 95% confidence interval for dose delivered per actuation is ±about 2.5%.

In some embodiments, upon nasal delivery of the pharmaceutical composition to the patient, less than about 20%, less than about 15%, less than about 10%, or less than about 5%, of the pharmaceutical composition leaves the nasal cavity via drainage into the nasopharynx or externally. In some embodiments, the plasma concentration versus time curve of the opioid antagonist in the patient has a Tₘₐₓ of less than 30 minutes, as provided above. In some embodiments, the plasma concentration versus time curve of the opioid antagonist in the patient has a Tₘₐₓ of about 30 minutes. In some embodiments, the plasma concentration versus time curve of the opioid antagonist in the patient has a Tₘₐₓ of about 25 minutes. In some embodiments, the plasma concentration versus time curve of the opioid antagonist in the patient has a Tₘₐₓ of about 20 minutes. In some embodiments, the plasma concentration versus time curve of the opioid antagonist in the patient has a Tₘₐₓ of about 15 minutes. In some embodiments, the plasma concentration versus time curve of the opioid antagonist in the patient has a Tₘₐₓ of about 10 minutes. In some embodiments, the plasma concentration versus time curve of the opioid antagonist in the patient has a Tₘₐₓ of about 5 minutes.

In some embodiments, delivery of the therapeutically effective amount to the patient, provides occupancy at Tₘₐₓ of the opioid antagonist at the opioid receptors in the respiratory control center of the patient of greater than about 90%, greater than about 95% or greater than about 99%, as provided above.

In some embodiments, the patient is free from respiratory depression for at least about 1 hour to at least about 8 hours following treatment comprising delivery of the therapeutically effective amount of the opioid antagonist, as provided above. In some embodiments, the patient is free from respiratory depression for at least about 3 hours to at least about 8 hours following treatment comprising delivery of the therapeutically effective amount of the opioid antagonist.

### Kits

Also provided are kits comprising a device described herein and written instructions for using the device. Also provided are kits comprising a device described herein and an opioid antagonist. In some embodiments, the kit further comprises written instructions.

### Pharmaceutical Compositions

Also provided are pharmaceutical compositions comprising naltrexone. In some embodiments, the pharmaceutical compositions comprise naltrexone and a pharmaceutically acceptable carrier. The carrier(s) must be “acceptable” in the sense of being compatible with the other ingredients of the formulation and not overly deleterious to the recipient thereof. Some embodiments of the present disclosure include a method of producing a pharmaceutical composition comprising admixing naltrexone and a pharmaceutically acceptable carrier. Pharmaceutical compositions are applied directly to the nasal cavity using the devices described herein. In the case of a spray, this may be achieved for example by means of a metering atomizing spray pump.

Liquid preparations include solutions, suspensions and emulsions, for example, water or water-propylene glycol solutions. Additional ingredients in liquid preparations may include: antimicrobial preservatives, such as benzalkonium chloride (which may also act as a cationic surfactant and/or a absorption enhancer), methylparaben, sodium benzoate, benzoic acid, phenyl ethyl alcohol, and the like, and mixtures thereof; surfactants such as Polysorbate 80 NF, polyoxyethylene 20 sorbitan monolaurate, polyoxyethylene (4) sorbitan monolaurate, polyoxyethylene 20 sorbitan monopalmitate, polyoxyethylene 20 sorbitan monostearate, polyoxyethylene (4) sorbitan monostearate, polyoxyethylene 20 sorbitan tristearate, polyoxyethylene (5) sorbitan monooleate, polyoxyethylene 20 sorbitan trioleate, polyoxyethylene 20 sorbitan monoisostearate, sorbitan monooleate, sorbitan monolaurate, sorbitan monopalmitate, sorbitan monostearate, sorbitan trilaurate, sorbitan trioleate, sorbitan tristearate, and the like, and mixtures thereof; a tonicity agent such as: dextrose, lactose, sodium chloride, calcium chloride, magnesium chloride, sorbitol, sucrose, mannitol, trehalose, raffinose, polyethylene glycol, hydroxyethyl starch, glycine, and the like, and mixtures thereof; and a suspending agent such as microcrystalline cellulose, carboxymethylcellulose sodium NF, polyacrylic acid, magnesium aluminum silicate, xanthan gum, and the like, and mixtures thereof.

Naltrexone can be formulated into pharmaceutical compositions using techniques well known to those in the art.

Naltrexone may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like, such as those pharmaceutically acceptable salts listed by Berge et al., Journal of Pharmaceutical Sciences, 66:1-19 (1977). The acid addition salts may be obtained as the direct products of compound synthesis. In the alternative, the free base may be dissolved in a suitable solvent containing the appropriate acid and the salt isolated by evaporating the solvent or otherwise separating the salt and solvent. Naltrexone may form solvates with standard low molecular weight solvents using methods known to the skilled artisan.

Accordingly, provided herein are pharmaceutical formulations for intranasal administration comprising, in an aqueous solution of not more than about 140 μL: between about 1 mg and about 10 mg of an opioid antagonist; between about 0.2 mg and about 1.2 mg of an isotonicity agent; between about 0.005 mg and about 0.015 mg of a compound which is a preservative, cationic surfactant, and/or absorption enhancer; between about 0.1 mg and about 0.5 mg of a stabilizing agent; an amount of an acid or base sufficient to achieve a pH of 3.5-5.5.

In some embodiments, said opioid antagonist is the only pharmaceutically active compound in said pharmaceutical composition.

In some embodiments, said opioid antagonist is naltrexone hydrochloride, or a hydrate thereof.

In some embodiments, said opioid antagonist is naltrexone hydrochloride.

The pharmaceutical formulation may comprise any of the amounts of naltrexone hydrochloride as provided above, for example, equivalent to about 1 mg to about 10 mg. In some embodiments, the therapeutically effective amount is equivalent to about 1 mg of naltrexone hydrochloride. In some embodiments, the therapeutically effective amount is equivalent to about 1.5 mg of naltrexone hydrochloride. In some embodiments, the therapeutically effective amount is equivalent to about 2 mg of naltrexone hydrochloride. In some embodiments, the therapeutically effective amount is equivalent to about 2.5 mg of naltrexone hydrochloride. In some embodiments, the therapeutically effective amount is equivalent to about 3 mg of naltrexone hydrochloride. In some embodiments, the therapeutically effective amount is equivalent to about 3.5 mg of naltrexone hydrochloride. In some embodiments, the therapeutically effective amount is equivalent to about 4 mg of naltrexone hydrochloride. In some embodiments, the therapeutically effective amount is equivalent to about 4.5 mg of naltrexone hydrochloride. In some embodiments, the therapeutically effective amount is equivalent to about 5 mg of naltrexone hydrochloride.

In some embodiments, the pharmaceutical composition is in an aqueous solution of about 100 μL.

In some embodiments, upon nasal delivery of the pharmaceutical composition to the patient, less than about 20%, less than about 15%, less than about 10%, or less than about 5%, of the pharmaceutical composition leaves the nasal cavity via drainage into the nasopharynx or externally, as provided above.

In some embodiments, the plasma concentration versus time curve of the opioid antagonist in the patient has a Tₘₐₓ of less than 30 minutes, as provided above. In some embodiments, the plasma concentration versus time curve of the opioid antagonist in the patient has a Tₘₐₓ of about 30 minutes. In some embodiments, the plasma concentration versus time curve of the opioid antagonist in the patient has a Tₘₐₓ of about 25 minutes. In some embodiments, the plasma concentration versus time curve of the opioid antagonist in the patient has a Tₘₐₓ of about 20 minutes. In some embodiments, the plasma concentration versus time curve of the opioid antagonist in the patient has a Tₘₐₓ of about 15 minutes. In some embodiments, the plasma concentration versus time curve of the opioid antagonist in the patient has a Tₘₐₓ of about 10 minutes. In some embodiments, the plasma concentration versus time curve of the opioid antagonist in the patient has a Tₘₐₓ of about 5 minutes.

In some embodiments, said device is actuatable with one hand.

In some embodiments, the delivery time is less than about 30 seconds. In some embodiments, the delivery time is less than about 25 seconds. In some embodiments, the delivery time is less than about 20 seconds. In some embodiments, the delivery time is less than about 15 seconds.

In some embodiments, the 90% confidence interval for dose delivered per actuation is ±about 2%. In some embodiments, the 95% confidence interval for dose delivered per actuation is ±about 2.5%.

In some embodiments, upon nasal delivery of the pharmaceutical composition to the patient, less than about 20%, less than about 15%, less than about 10%, or less than about 5%, of the pharmaceutical composition leaves the nasal cavity via drainage into the nasopharynx or externally, as provided above.

In some embodiments, the plasma concentration versus time curve of the opioid antagonist in the patient has a Tₘₐₓ of less than 30 minutes, as provided above. In some embodiments, the plasma concentration versus time curve of the opioid antagonist in the patient has a Tₘₐₓ of about 30 minutes. In some embodiments, the plasma concentration versus time curve of the opioid antagonist in the patient has a Tₘₐₓ of about 25 minutes. In some embodiments, the plasma concentration versus time curve of the opioid antagonist in the patient has a Tm Tₘₐₓ ax of about 20 minutes. In some embodiments, the plasma concentration versus time curve of the opioid antagonist in the patient has a Tₘₐₓ of about 15 minutes. In some embodiments, the plasma concentration versus time curve of the opioid antagonist in the patient has a Tₘₐₓ of about 10 minutes. In some embodiments, the plasma concentration versus time curve of the opioid antagonist in the patient has a Tₘₐₓ of about 5 minutes.

In some embodiments, delivery of the therapeutically effective amount to the patient, provides occupancy at Tₘₐₓ of the opioid antagonist at the opioid receptors in the respiratory control center of the patient of greater than about 90%, greater than about 95% or greater than about 99%, as provided above.

In some embodiments, the patient is free from respiratory depression for at least about 1 hour to at least about 8 hours following treatment comprising delivery of the therapeutically effective amount of the opioid antagonist, as provided above. In some embodiments, the patient is free from respiratory depression for at least about 3 hours to at least about 8 hours following treatment comprising delivery of the therapeutically effective amount of the opioid antagonist.

Also provided herein are pharmaceutical formulations for intranasal administration comprising, in an aqueous solution of not more than about 140 μL: about 1 mg or about 10 mg naltrexone hydrochloride or a hydrate thereof; between about 0.2 mg and about 1.2 mg of an isotonicity agent; between about 0.005 mg and about 0.015 mg of a compound which is a preservative, cationic surfactant, and/or absorption enhancer; between about 0.1 mg and about 0.5 mg of a stabilizing agent; an amount of hydrochloric acid or sodium hydroxide sufficient to achieve a pH of 3.5-5.5.

In some embodiments, the isotonicity agent is NaCl; the compound which is a preservative, cationic surfactant, and/or absorption enhancer is benzalkonium chloride; the stabilizing agent is disodium edetate; and the acid is hydrochloric acid or the base is sodium hydroxide.

In some embodiments, the pharmaceutical formulation comprises: about 2 mg or about 3 mg or about 4 mg naltrexone hydrochloride; about 0.74 mg NaCl; about 0.01 mg benzalkonium chloride; about 0.2 mg disodium edetate; and an amount of hydrochloric acid or sodium hydroxide sufficient to achieve a pH of 3.5-5.5.

In some embodiments, the pharmaceutical formulation comprises about 4 mg naltrexone hydrochloride or a hydrate thereof. In some embodiments, the pharmaceutical formulation comprises about 3 mg naltrexone hydrochloride or a hydrate thereof.

Also provided herein are pharmaceutical formulations for intranasal administration comprising, in an aqueous solution of about 100 μL: about 3 mg naltrexone hydrochloride or a hydrate thereof between about 0.2 mg and about 1.2 mg of an isotonicity agent; between about 0.005 mg and about 0.015 mg of a compound which is a preservative, cationic surfactant, and/or absorption enhancer; between about 0.1 mg and about 0.5 mg of a stabilizing agent; an amount of an acid or base sufficient to achieve a pH of 3.5-5.5.

In some embodiments, the pharmaceutical formulation comprises: about 1 mg to about 10 mg naltrexone hydrochloride; about 0.74 mg NaCl; about 0.01 mg benzalkonium chloride; about 0.2 mg disodium edetate; and an amount of hydrochloric acid or sodium hydroxide sufficient to achieve a pH of 3.5-5.5.

Also provided herein are pharmaceutical formulations for intranasal administration comprising, in an aqueous solution of about 100 μL: about 3 mg naltrexone hydrochloride or a hydrate thereof between about 0.2 mg and about 1.2 mg of an isotonicity agent; between about 0.005 mg and about 0.015 mg of a compound which is a preservative, cationic surfactant, and/or absorption enhancer; between about 0.1 mg and about 0.5 mg of a stabilizing agent; an amount of an acid or base sufficient to achieve a pH of 3.5-5.5.

In some embodiments, the pharmaceutical formulation comprises: about 3 mg naltrexone hydrochloride; about 0.74 mg NaCl; about 0.01 mg benzalkonium chloride; about 0.2 mg disodium edetate; and an amount of hydrochloric acid or sodium hydroxide sufficient to achieve a pH of 3.5-5.5.

Also provided herein are pharmaceutical formulations for intranasal administration comprising, in an aqueous solution of about 100 μL: about 1 to about 10 mg naltrexone hydrochloride or a hydrate thereof; between about 0.2 mg and about 1.2 mg of an isotonicity agent; between about 0.005 mg and about 0.015 mg of a compound which is a preservative, cationic surfactant, and/or absorption enhancer; between about 0.1 mg and about 0.5 mg of a stabilizing agent; an amount of an acid or base sufficient to achieve a pH of 3.5-5.5.

Provided are devices adapted for nasal delivery of a pharmaceutical composition to a patient, comprising a therapeutically effective amount of an opioid antagonist selected from naltrexone and pharmaceutically acceptable salts thereof, wherein the device is optionally pre-primed, and wherein the therapeutically effective amount, is equivalent to about 1 mg to about 10 mg of naltrexone hydrochloride, as provided above. In some embodiments, the therapeutically effective amount is equivalent to about 1 mg of naltrexone hydrochloride. In some embodiments, the therapeutically effective amount is equivalent to about 1.5 mg of naltrexone hydrochloride. In some embodiments, the therapeutically effective amount is equivalent to about 2 mg of naltrexone hydrochloride. In some embodiments, the therapeutically effective amount is equivalent to about 2.5 mg of naltrexone hydrochloride. In some embodiments, the therapeutically effective amount is equivalent to about 3 mg of naltrexone hydrochloride. In some embodiments, the therapeutically effective amount is equivalent to about 3.5 mg of naltrexone hydrochloride. In some embodiments, the therapeutically effective amount is equivalent to about 4 mg of naltrexone hydrochloride. In some embodiments, the therapeutically effective amount is equivalent to about 4.5 mg of naltrexone hydrochloride. In some embodiments, the therapeutically effective amount is equivalent to about 5 mg of naltrexone hydrochloride.

In some embodiments, the pharmaceutical composition comprises a solution prepared from naltrexone hydrochloride. In some embodiments, the pharmaceutical composition further comprises one or more excipients selected from water and NaCl. In some embodiments, the pharmaceutical composition is substantially free of antimicrobial preservatives. In some embodiments, the device is substantially free of benzalkonium chloride, methylparaben, sodium benzoate, benzoic acid, phenyl ethyl alcohol. In some embodiments, the device is filled with the pharmaceutical composition in a sterile environment. In some embodiments, the pharmaceutical composition is storage-stable for about twelve months at about 25° C. In some embodiments, the pharmaceutical composition comprises less than 0.1% w/w antimicrobial preservatives. In some embodiments, the pharmaceutical composition comprises 0.01% w/w or less antimicrobial preservatives. In some embodiments, the pharmaceutical composition comprises 0.01% w/w-0.001% w/w antimicrobial preservatives. In some embodiments, the pharmaceutical composition comprises less than 0.001% w/w antimicrobial preservatives.

Also provided are embodiments wherein any embodiment above may be combined with any one or more of these embodiments, provided the combination is not mutually exclusive.

### Indications

Also provided are devices for use in treating opioid overdose and symptoms thereof and methods of using the devices. Naltrexone prevents or reverses the effects of opioids including respiratory depression, sedation and hypotension.

Accordingly, also provided herein are methods of treating opioid overdose or a symptom thereof, comprising nasally administering to a patient in need thereof a therapeutically effective amount of an opioid antagonist selected from naltrexone and pharmaceutically acceptable salts thereof, wherein said therapeutically effective amount is equivalent to about 1 mg to about 10 mg of naltrexone hydrochloride or a hydrate thereof. In some embodiments, the therapeutically effective amount of an opioid antagonist selected from naltrexone and pharmaceutically acceptable salts thereof is delivered in not more than about 140 μL of an aqueous carrier solution.

In some embodiments, also provided herein are methods of treating opioid overdose or a symptom thereof, comprising nasally administering to a patient in need thereof a therapeutically effective amount of an opioid antagonist selected from naltrexone and pharmaceutically acceptable salts thereof, wherein said therapeutically effective amount is equivalent to about 1 mg to about 10 mg of naltrexone hydrochloride or a hydrate thereof in not more than about 140 μL of an aqueous carrier solution.

In some embodiments, said opioid antagonist is the only pharmaceutically active compound in said pharmaceutical composition.

In some embodiments, said opioid antagonist is naltrexone hydrochloride.

In some embodiments, said pharmaceutical composition comprises a solution of naltrexone hydrochloride, or a hydrate thereof.

In some embodiments, said patient is an opioid overdose patient or a suspected opioid overdose patient.

In some embodiments, said patient is in a lying, supine, or recovery position. In some embodiments, said patient is in a lying position. In some embodiments, said patient is in a supine position. In some embodiments, said patient is in a recovery position.

In some embodiments, said therapeutically effective amount of an opioid antagonist is delivered by an untrained individual.

In some embodiments, said therapeutically effective amount is equivalent to about 1 mg to about 10 mg of naltrexone hydrochloride, as provided above. In some embodiments, the therapeutically effective amount is equivalent to about 1 mg of naltrexone hydrochloride. In some embodiments, the therapeutically effective amount is equivalent to about 1.5 mg of naltrexone hydrochloride. In some embodiments, the therapeutically effective amount is equivalent to about 2 mg of naltrexone hydrochloride. In some embodiments, the therapeutically effective amount is equivalent to about 2.5 mg of naltrexone hydrochloride. In some embodiments, the therapeutically effective amount is equivalent to about 3 mg of naltrexone hydrochloride. In some embodiments, the therapeutically effective amount is equivalent to about 3.5 mg of naltrexone hydrochloride. In some embodiments, the therapeutically effective amount is equivalent to about 4 mg of naltrexone hydrochloride. In some embodiments, the therapeutically effective amount is equivalent to about 4.5 mg of naltrexone hydrochloride. In some embodiments, the therapeutically effective amount is equivalent to about 5 mg of naltrexone hydrochloride.

In some embodiments, said symptom is chosen from respiratory depression and central nervous system depression.

In some embodiments, said patient exhibits any of unresponsiveness to stimulus, unconsciousness, stopped breathing; erratic or stopped pulse, choking or gurgling sounds, blue or purple fingernails or lips, slack or limp muscle tone, contracted pupils, and vomiting.

In some embodiments, said patient is not breathing.

In some embodiments, said patient is in a lying, supine, or recovery position.

In some embodiments, said patient is in a lying position.

In some embodiments, said patient is in a supine position.

In some embodiments, said patient is a recovery position.

In some embodiments, said therapeutically effective amount is equivalent to about 2 mg to about 10 mg of naltrexone hydrochloride.

In some embodiments, said therapeutically effective amount is equivalent to an amount chosen from about 1 mg naltrexone hydrochloride, about 1.5 mg naltrexone hydrochloride, about 2 mg of naltrexone hydrochloride, about 2.5 mg of naltrexone hydrochloride, about 3 mg naltrexone hydrochloride, about 3.5 mg naltrexone hydrochloride, about 4 mg naltrexone hydrochloride, about 4.5 mg naltrexone hydrochloride and about 5 mg naltrexone hydrochloride.

In some embodiments, said therapeutically effective amount is equivalent to about 2 mg of naltrexone hydrochloride.

In some embodiments, said therapeutically effective amount is equivalent to about 3 mg of naltrexone hydrochloride.

In some embodiments, said therapeutically effective amount is equivalent to about 4 mg of naltrexone hydrochloride.

In some embodiments, said opioid antagonist is the only pharmaceutically active compound in said pharmaceutical composition.

In some embodiments, said nasally administering is accomplished using a pre-primed device adapted for nasal delivery of a pharmaceutical composition. In some embodiments, said nasally administering is accomplished using a multi-dose device adapted for nasal delivery of a pharmaceutical composition.

In some embodiments, upon nasal delivery of the pharmaceutical composition to the patient, less than about 20%, less than about 15%, less than about 10%, or less than about 5%, of the pharmaceutical composition leaves the nasal cavity via drainage into the nasopharynx or externally, as provided above.

In some embodiments, the plasma concentration versus time curve of the opioid antagonist in the patient has a Tₘₐₓ of less than 30 minutes, as provided above. In some embodiments, the plasma concentration versus time curve of the opioid antagonist in the patient has a Tₘₐₓ of about 30 minutes. In some embodiments, the plasma concentration versus time curve of the opioid antagonist in the patient has a Tₘₐₓ of about 25 minutes. In some embodiments, the plasma concentration versus time curve of the opioid antagonist in the patient has a Tₘₐₓ of about 20 minutes. In some embodiments, the plasma concentration versus time curve of the opioid antagonist in the patient has a Tₘₐₓ of about 15 minutes. In some embodiments, the plasma concentration versus time curve of the opioid antagonist in the patient has a Tₘₐₓ of about 10 minutes. In some embodiments, the plasma concentration versus time curve of the opioid antagonist in the patient has a Tₘₐₓ of about 5 minutes.

In some embodiments, said opioid overdose symptom is selected from: respiratory depression, central nervous system depression, and cardiovascular depression.

In some embodiments, said opioid overdose symptom is respiratory depression induced by opioids.

In some embodiments, said respiratory depression is caused by the illicit use of opioids or by an accidental misuse of opioids during medical opioid therapy.

In some embodiments, said respiratory depression is induced by opioids selected from: natural and synthetic narcotics, propoxyphene, methadone, nalbuphine, pentazocine and butorphanol.

In some embodiments, said respiratory depression is induced by an opioid selected from codeine, morphine, methadone, fentanyl, oxycodone HCl, hydrocodone bitartrate, hydromorphone, oxymorphone, meperidine, propoxyphene, opium, heroin, tramadol, and tapentadol.

In some embodiments, the patient is free from respiratory depression for at least about 1 hour to at least about 8 hours following treatment comprising delivery of the therapeutically effective amount of the opioid antagonist, as provided above. In some embodiments, the patient is free from respiratory depression for at least about 3 hours to at least about 8 hours following treatment comprising delivery of the therapeutically effective amount of the opioid antagonist.

Also provided are embodiments wherein any embodiment above may be combined with any one or more of these embodiments, provided the combination is not mutually exclusive.

Also provided are the devices, pharmaceutical compositions, kits, and methods of treatment described herein for use in the treatment of an opioid overdose symptom selected from: respiratory depression, postoperative opioid respiratory depression, altered level consciousness, miotic pupils, cardiovascular depression, hypoxemia, acute lung injury, aspiration pneumonia, sedation, and hypotension. Also provided are the devices, pharmaceutical compositions, kits, and methods of treatment described herein for use in the reversal of respiratory depression induced by opioids.

Also provided are the devices, pharmaceutical compositions, kits, and methods of treatment described herein for use in the complete or partial reversal of narcotic depression, including respiratory depression, induced by opioids selected from: natural and synthetic narcotics, propoxyphene, methadone, nalbuphine, pentazocine and butorphanol. In some embodiments, narcotic depression, including respiratory depression, is induced by an opioid agonist selected from codeine, morphine, methadone, fentanyl, oxycodone HCl, hydrocodone bitartrate, hydromorphone, oxymorphone, meperidine, propoxyphene, opium, heroin, tramadol, and tapentadol.

Also provided are devices, pharmaceutical formulations, and kits for, and methods of, treating opioid overdose or a symptom thereof, comprising nasally administering to a patient in need thereof a therapeutically effective amount of an opioid antagonist selected from naltrexone and pharmaceutically acceptable salts thereof, wherein the therapeutically effective amount is equivalent to about 1 mg to about 10 mg of naltrexone hydrochloride. In some embodiments, the patient is not breathing. Also provided are devices adapted for nasal delivery of a pharmaceutical composition to a patient, comprising a therapeutically effective amount of an opioid antagonist selected from naltrexone and pharmaceutically acceptable salts thereof, wherein the device is pre-primed, and wherein the therapeutically effective amount, is equivalent to about 1 mg to about 10 mg of naltrexone hydrochloride, as provided above.

In some embodiments, the therapeutically effective amount is equivalent to about 1 mg of naltrexone hydrochloride. In some embodiments, the therapeutically effective amount is equivalent to about 1.5 mg of naltrexone hydrochloride. In some embodiments, the therapeutically effective amount is equivalent to about 2 mg of naltrexone hydrochloride. In some embodiments, the therapeutically effective amount is equivalent to about 2.5 mg of naltrexone hydrochloride. In some embodiments, the therapeutically effective amount is equivalent to about 3 mg of naltrexone hydrochloride. In some embodiments, the therapeutically effective amount is equivalent to about 3.5 mg of naltrexone hydrochloride. In some embodiments, the therapeutically effective amount is equivalent to about 4 mg of naltrexone hydrochloride. In some embodiments, the therapeutically effective amount is equivalent to about 4.5 mg of naltrexone hydrochloride. In some embodiments, the therapeutically effective amount is equivalent to about 5 mg of naltrexone hydrochloride.

In some embodiments, the opioid antagonist is the only pharmaceutically active compound in pharmaceutical composition. In some embodiments, the opioid antagonist is naltrexone hydrochloride. In some embodiments, the opioid antagonist is anhydrous naltrexone hydrochloride. In some embodiments, the pharmaceutical composition comprises a solution of naltrexone hydrochloride. In some embodiments, the nasally administering is accomplished using a device described herein. In some embodiments, the opioid overdose symptom is selected from: respiratory depression, postoperative opioid respiratory depression, altered level consciousness, miotic pupils, cardiovascular depression, hypoxemia, acute lung injury, aspiration pneumonia, sedation, and hypotension. In some embodiments, the opioid overdose symptom is respiratory depression induced by opioids. In some embodiments, the respiratory depression is caused by the illicit use of opioids or by an accidental misuse of opioids during medical opioid therapy.

Also provided are devices, kits, and pharmaceutical formulations for, and methods of, reversing the psychotomimetic and dysphoric effects of agonist-antagonists such as pentazocine, comprising nasally administering to a patient in need thereof a therapeutically effective amount of an opioid antagonist selected from naltrexone and pharmaceutically acceptable salts thereof, wherein the therapeutically effective amount is equivalent to about 1 mg to about 10 mg of naltrexone hydrochloride, as provided above.

Also provided are devices, kits, and pharmaceutical formulations for, and methods of, diagnosis of suspected acute opioid over-dosage, comprising nasally administering to a patient in need thereof a therapeutically effective amount of an opioid antagonist selected from naltrexone and pharmaceutically acceptable salts thereof, wherein the therapeutically effective amount is equivalent to about 1 mg to about 10 mg of naltrexone hydrochloride, as provided above.

Also provided are devices, kits, and pharmaceutical formulations for, and methods of, treating opioid addiction, comprising nasally administering to a patient in need thereof a therapeutically effective amount of an opioid antagonist selected from naltrexone and pharmaceutically acceptable salts thereof, wherein the therapeutically effective amount is equivalent to about 1 mg to about 10 mg of naltrexone hydrochloride, as provided above.

Also provided are devices, kits, and pharmaceutical formulations for, and methods of, treating septic shock, comprising nasally administering to a patient in need thereof a therapeutically effective amount of an opioid antagonist selected from naltrexone and pharmaceutically acceptable salts thereof, wherein the therapeutically effective amount is equivalent to about 1 mg to about 10 mg of naltrexone hydrochloride, as provided above.

Also provided are devices, kits, and pharmaceutical formulations for, and methods of, treating opioid overdose or a symptom thereof, reversing the psychotomimetic and dysphoric effects of agonist-antagonists such as pentazocine, diagnosing suspected acute opioid over-dosage, treating opioid addiction, or treating septic shock, comprising nasally administering to a patient in need thereof a therapeutically effective amount of an opioid antagonist, wherein the therapeutically effective amount is about 1 mg to about 10 mg, as provided above.

However, the efficacy of oral naltrexone is hampered by slow onset and low bioavailability, and the injectable form of naltrexone presents itself with the obvious difficulties associated with needles including, for example, the need for administration by a practitioner at regularly scheduled intervals. Thus, intranasal administration of naltrexone, and use of absorption enhancers, in a single (optionally pre-primed) or multi-use nasal spray pump should significantly improve results. An intranasal formulation of naltrexone absorbs quickly, providing fast onset of action and high bioavailability without the use of needles.

Also provided are pharmaceutical compositions in a device adapted for nasal delivery to a subject suffering from an addiction or disorder associated with reward-based behavior, comprising a therapeutically effective amount of the opioid antagonist naltrexone and pharmaceutically acceptable salts thereof. In some embodiments, the device is pre-primed. In some embodiments, the device can be primed before use. In some embodiments, the device can be actuated with one hand.

The reward system is a group of neural structures responsible for incentive salience (i.e., “wanting” or desire), pleasure (i.e., “liking” or hedonic value), and positive reinforcement (i.e., learning). Reward is the attractive and motivational property of a stimulus that induces appetitive behavior—also known as approach behavior—and consummatory behavior. See, Wolfram Shultz, Neuronal Reward and Decision Signals: From Theories to Data, Physiology Review 95:853-951, 2015. Addictive drugs and addictive behaviors are rewarding and reinforcing (i.e., addictive) due to their effects on the dopamine reward pathway.

Dopaminergic pathways, sometimes called dopaminergic projections, are neural pathways in the brain that transmit the neurotransmitter dopamine from one region of the brain to another. There are four major dopaminergic pathways: mesolimbic, mesocortical, nigrostriatal, and tuberoinfundibular. In particular, the mesolimbic pathway associated cognitive processes include reward-related cognition (e.g., incentive salience, pleasure, and positive reinforcement).

Opiate receptors are integral components of the brain's reward circuitry. Both exogenous opioids (drugs) and endogenous opioid peptides (endorphins and enkephalins), by dint of binding to and activating these opiate receptors, activate the brain's reward system by flooding the system with dopamine. Alcohol consumption, and certain pleasurable behaviors ranging from eating calorie dense foods to intense exercise and sexual activity also cause a release of endogenous opioids, resulting in the activation of this reward circuitry. The interaction between opioids and the dopaminergic system is involved in addiction, tolerance, and withdrawal symptoms. The relevant interactions occur along the mesolimbic projection, particularly in the ventral tegmental area (VTA) and nucleus accumbens (NA). The neurotransmitter dopamine is present in regions of the brain that regulate movement, emotion, cognition, motivation, and feelings of pleasure. Overstimulation of this system, which rewards behaviors, produces the euphoric effects sought by individuals who misuse drugs and alcohol and teaches them to repeat the behavior. Since each class of opioid receptor has a unique effect on the brain, the multitude of classes allows opioids to have a wide range of effects in the body.

Behavioral addictions—sometimes referred to as impulse control disorders—are a form of addiction that involves a compulsion to engage in a rewarding non-drug-related behavior despite any negative consequences to the person's physical, mental, social or financial well-being. The type of excessive behaviors identified as being addictive include gambling, food, sex, use of pornography, use of computers, playing video games, use of the internet, exercise, and shopping. As detailed above, these behaviors can trigger the release of endogenous opioid peptides leading to activation of the brain's reward circuitry, much like is seen following drug and alcohol consumption.

### OTHER EMBODIMENTS

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

Also provided are embodiments wherein any embodiment above can be combined with any one or more of these embodiments, provided the combination is not mutually exclusive. Also provided herein are uses in the treatment of indications or one or more symptoms thereof as disclosed herein, and uses in the manufacture of medicaments for the treatment of indications or one or more symptoms thereof as disclosed herein, equivalent in scope to any embodiment disclosed above, or any combination thereof that is not mutually exclusive. The methods and uses may employ any of the devices disclosed herein, or any combination thereof that is not mutually exclusive, or any of the pharmaceutical formulations disclosed herein, or any combination thereof that is not mutually exclusive.

### EXAMPLES

The following examples are included to demonstrate preferred embodiments of the disclosure. The following examples are presented only by way of illustration and to assist one of ordinary skill in using the disclosure. The examples are not intended in any way to otherwise limit the scope of the disclosure. Those of skill in the art should, in light of the present disclosure, appreciate that many changes can be made in the specific embodiments which are disclosed and still obtain a like or similar result without departing from the spirit and scope of the disclosure.

### Example 1

Study Goals. The purpose of this clinical study was twofold: to determine and compare the pharmacokinetics of two intranasal formulations (4 mg with and without Intravail®), an oral formulation (50 mg tablet) and a 2-mg intramuscular dose of naltrexone; and to determine the safety of intranasal naltrexone, particularly with respect to nasal irritation, such as inflammation (erythema, edema, and erosion) and bleeding. To that end, the study's primary endpoints were the pharmacokinetic parameters (Cₘₐₓ, Tₘₐₓ, AUC₀₋ₜ, and AUC_{0-inf}) produced by the IN, oral and IM doses of naltrexone. Secondary endpoints included adverse events (AEs), vital signs (heart rate, sitting blood pressure, and respiration rate), electrocardiogram (ECG), clinical laboratory changes and nasal irritation using the nasal irritation scale.

Study Design.

Fourteen healthy volunteers were enrolled and completed all study drug administrations and blood collections for PK assessments. This was an in-patient open-label, crossover study involving approximately 14 healthy volunteers. Each subject received each naltrexone treatment: 4 mg intranasal (IN; one 0.1 mL spray of a 40 mg/mL solution in one nostril), 4 mg plus 0.25% Intravail® IN (one 0.1 mL spray of a 40 mg/mL solution containing 0.25% Intravail® in one nostril), 2 mg intramuscular (IM), and 50 mg oral tablet (PO). Subjects stayed in the in-patient facility for 13 days to complete the entire study. Subjects were called 3 to 5 days after discharge to inquire concerning AEs and concomitant medications since discharge. Informed consent was obtained from all subjects, and all were screened for eligibility to participate in the study, including medical history, physical examination, clinical chemistry, coagulation markers, hematology, infectious disease serology, urinalysis, urine drug and alcohol toxicology screen, vital signs and ECG.

On the day after clinic admission, subjects were administered study drug with a 3-day washout period between doses until all treatments had been administered. Blood was collected for analysis prior to dosing and approximately 2.5, 5, 10, 15, 20, 30, 45, 60 minutes and 2, 3, 4, 6, 8, 12, 16, 24, 30, 36, and 48 hours after study drug administration. On days of study drug administration, a 12-lead ECG was performed approximately 1 hour prior to dosing and at approximately 1 and 4 hours post-dose. Vital signs were measured pre-dose and about 1 and 4 hours post-dose.

On dosing days, the order of assessments was ECG, vital signs, then PK blood collection when scheduled at the same nominal times. The target time of the PK blood collection was considered the most critical and if the collection was more than ±1 minute from the scheduled time for the first 60 minutes of collections or more than ±5 minutes for the scheduled time points thereafter, this was considered a protocol deviation. ECG and vital signs were collected within the 10-minute period before the nominal time of blood collections. At screening, admission, and discharge, ECG, and vital signs were checked once per day. Vital signs were also checked once on the day after naltrexone administration. Clinical laboratory measurements were repeated after the last PK blood draw prior to clinic discharge. AEs were assessed by spontaneous reports by subjects, by examination of the nasal mucosa, by measuring vital signs, ECG, and clinical laboratory parameters.

Inclusion and Exclusion Criteria:

1. Males and females 18 to 55 years of age, inclusive were included in this study. Written informed consent was required. Subject had to:
have body mass index (BMI) ranging from 18 to 30 kg/m², inclusive;
have adequate venous access;
have no clinically significant concurrent medical conditions determined by medical history, physical examination, clinical laboratory examination, vital signs, and 12-lead ECG;
agree to use an acceptable method of contraception, other than oral contraceptives, throughout the study and for 90 days after the last study drug administration (30 days for women); and
agree not to ingest alcohol, drinks containing xanthine >500 mg/day (e.g., Coca-Cola®, tea, coffee, etc.), or grapefruit/grapefruit juice or participate in strenuous exercise 72 hours prior to admission through the last blood draw of the study.

Exclusion criteria included:
any IN conditions including abnormal nasal anatomy, nasal symptoms (i.e., blocked and/or runny nose, nasal polyps, etc.);
having a product sprayed into the nasal cavity prior to screening and drug administration;
having been administered an investigational drug within 30 days prior to Day −1;
having taken prescribed or over-the-counter medications, dietary supplements, herbal products, vitamins, or recent use of opioid analgesics for pain relief (within 14 days of last use of any of these products);
a positive urine drug test for alcohol, opioids, cocaine, amphetamine, methamphetamine, benzodiazepines, tetrahydrocannabinol (THC), barbiturates, or methadone at screening or admission;
previous or current opioid, alcohol, or other drug dependence (excluding nicotine and caffeine), based on medical history;
consumption of greater than 20 cigarettes per day on average, in the month prior to screening, or would be unable to abstain from smoking (or use of any nicotine-containing substance) for at least one hour prior to and 2 hours after naltrexone dosing;
systolic blood pressure less than 90 mm Hg or greater than 140 mm Hg; diastolic blood pressure less than 55 mmHg or greater than 90 mmHg; respiratory rate less than 8 respirations per minute or greater than 20 respirations per minute;
on standard 12-lead ECG, a QTcF interval >440 msec for males and >450 msec for females; significant acute or chronic medical disease (investigator judgment);
a likely need for concomitant medication treatment during the study;
donated or received blood or underwent plasma or platelet apheresis within the 60 days prior to Day −1;
female who is pregnant, breast feeding, or plans to become pregnant during the study period or within 30 days after the last naltrexone administration;
positive test for hepatitis B surface antigen (HBsAg), hepatitis C virus antibody (HCVAb) or human immunodeficiency virus antibody (HIVAb) at screening;
current or recent (within 7 days prior to screening) upper respiratory tract infection; and
abnormal liver function test (ALT, AST, total bilirubin)>1.5 times upper limit of normal.

Study Drugs and Dosing.

Naltrexone hydrochloride (HCl) was obtained from Mallinckrodt Pharmaceuticals. The IN (40 mg/mL) formulations were made by the staff pharmacist at Vince & Associates; the vehicle for the IN formulations was sterile water for injection. The IM formulation (2 mg/mL) was made by the staff pharmacist at Vince & Associates; the vehicle was sterile saline for injection. IN naltrexone was administered using an Aptar™ multi-dose device with the subject in a reclined position (approximately 45 degrees). The subject was instructed not to breathe through the nose when the IN dose of naltrexone was administered. Naltrexone HCl for the IM injection was administered with a 23-g needle as a single 1-mL injection into the gluteus maximus muscle. Naltrexone HCl for oral administration (50 mg tablet) was sourced from a commercial supplier. and administered with 240 mL water.

Naltrexone was administered on Days 1, 4, 7, and 10, in the following order: 4 mg naltrexone IN, 4 mg naltrexone plus Intravail® IN, 2 mg IM, and 50 mg oral. Subjects stayed in the in-patient facility for 13 days to complete the entire study and were discharged 2 days after the fourth dose.

PK Assessments.

Blood (4 mL) was collected in sodium heparin containing tubes for PK analysis prior to dosing and 2.5, 5, 10, 1.5, 20, 30, 45, 60 minutes and 2, 3, 4, 6, 8, 12, 16, 24, 30, 36, and 48 hours after the start of study drug administration. Plasma was separated from whole blood and stored frozen at <20° C. until assayed. Naltrexone and 6β-naltrexol plasma concentrations were determined by liquid chromatography with tandem mass spectrometry at XenoBiotic Laboratories, Inc., Plainsboro, N.J.

Safety Assessments.

Heart rate, blood pressure, and respiration rate were recorded approximately 1 hour before naltrexone dosing and approximately 1 and 4 hours after dosing. A 12-lead echocardiogram (ECG) was obtained about 1 hour before and 1 and 4 hours after each naltrexone dose. ECG and vital signs was performed within the 10-minute period before the nominal time for post-dose blood collections. Adverse events (AEs) were recorded from the start of study drug administration until clinic discharge. AEs were recorded relative to each dosing session to attempt to establish a relationship between the AE and type of naltrexone dose administered. An examination of the nasal passage was conducted at Day −1 to establish eligibility and at pre-dose, 5 minutes, 30 minutes, 60 minutes, 4 hours, and 24 hours post IN naltrexone administration to evaluate evidence of irritation to the nasal mucosa after IN administration only.

Analysis.

Non-compartmental PK parameters of naltrexone and 6β-naltrexol, including, Tₘₐₓ, AUC₀₋ₜ, and AUC_{0-inf}, t_{1/2}, λ_{z}, and apparent clearance (CL/F, naltrexone only), was determined. Pharmacokinetic parameters (Cₘₐₓ, Tₘₐₓ, and AUCs) for IN and PO naltrexone were compared with those for IM naltrexone. Dose-adjusted values for AUCs and Cₘₐₓ were calculated. The relative extent of IN and PO absorption (IN and PO versus IM) will be estimated from the dose-corrected AUCs. Within an analysis of variance (ANOVA) framework, comparisons of ln-transformed PK parameters (Cₘₐₓ and AUC) for IN and PO versus IM naltrexone treatments were performed. The 90% confidence interval for the ratio (IN/IM and PO/IM) of the geometric least squares means of AUC and Cₘₐₓ parameters were constructed for comparison of each treatment with IM naltrexone. These 90% confidence intervals (CIs) were obtained by exponentiation of the 90% CIs for the difference between the least squares means based upon an ln scale.

Naltrexone Results.

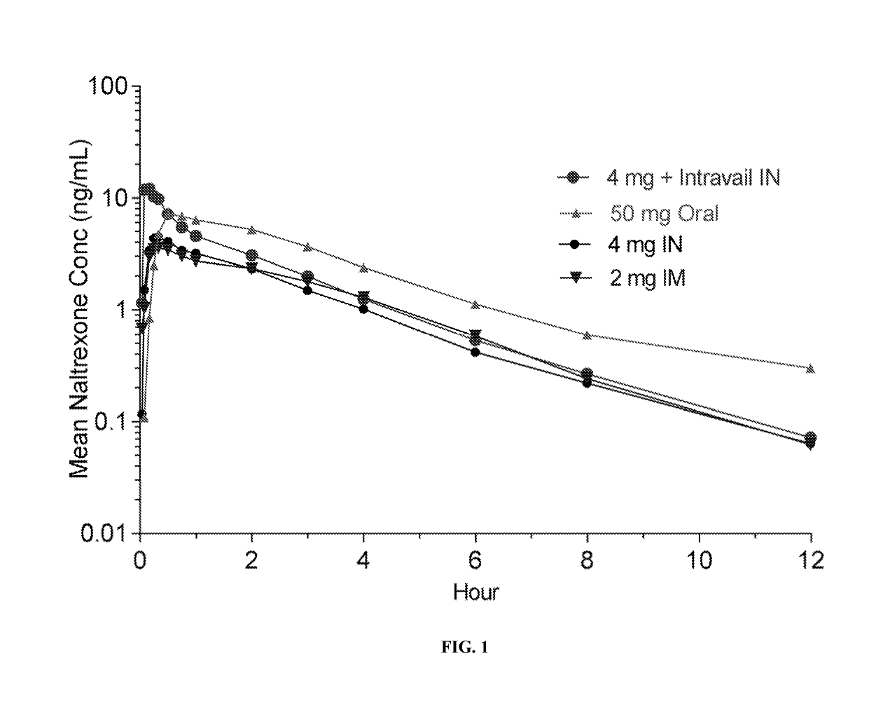

Results are shown below in Tables 1-3 and at FIG. 1.

TABLE 1
Mean (SD) concentrations of naltrexone following a single IN, IM or
oral administration to healthy subjects.

| | Treatment | | | | | | | |
|---|---|---|---|---|---|---|---|---|
| | | | 4 mg IN + 0.25% | | | | | |
| Hour | 4 mg IN | | Intravail ® | | 2 mg IN | | 50 mg Oral | |
| 0 | 0 | 0 | 0 | 0 | 0 | 0 | 0 | 0 |
| 0.042 | 0.117 | (0.17) | 1.15 | (0.919) | 0.678 | (1.69) | 0 | 0 |
| 0.083 | 1.51 | (1.62) | 11.9 | (9.69) | 1.04 | (1.26) | 0.109 | (0.232) |
| 0.17 | 3.4 | (3.86) | 12.1 | (6.36) | 2.97 | (2) | 0.851 | (1.5) |
| 0.25 | 4.36 | (3.71) | 10.4 | (3.93) | 3.45 | (1.58) | 2.5 | (3.54) |
| 0.33 | 4.46 | (3.62) | 9.81 | (2.41) | 3.58 | (1.46) | 4.75 | (4.71) |
| 0.5 | 4.08 | (1.99) | 7.19 | (2.08) | 3.43 | (1.06) | 7.16 | (4.69) |
| 0.75 | 3.39 | (1.46) | 5.46 | (1.48) | 3.02 | (0.749) | 6.9 | (3.54) |
| 1 | 3.19 | (1.51) | 4.55 | (1.78) | 2.73 | (0.676) | 6.34 | (2.78) |
| 2 | 2.33 | (0.832) | 3.07 | (1.27) | 2.35 | (0.698) | 5.22 | (1.73) |
| 3 | 1.5 | (0.633) | 2 | (0.885) | 1.79 | (0.491) | 3.66 | (1.76) |
| 4 | 1.02 | (0.369) | 1.25 | (0.465) | 1.3 | (0.341) | 2.39 | (1.16) |
| 6 | 0.418 | (0.193) | 0.536 | (0.188) | 0.584 | (0.185) | 1.13 | (0.462) |
| 8 | 0.22 | (0.0941) | 0.267 | (0.105) | 0.242 | (0.0803) | 0.596 | (0.426) |
| 12 | 0.0641 | (0.021) | 0.0726 | (0.028) | 0.0626 | (0.0269) | 0.3 | (0.183) |
| 16 | 0.0214 | (0.0131) | 0.0226 | (0.0165) | 0.0101 | (0.0132) | 0.141 | (0.104) |
| 24 | 0.00462 | (0.0117) | 0 | 0 | 0 | 0 | 0.0657 | (0.0528) |
| 30 | 0.00187 | (0.00674) | 0 | 0 | 0 | 0 | 0.0345 | (0.0224) |
| 36 | 0 | 0 | 0 | 0 | 0 | 0 | 0.0207 | (0.0255) |
| 48 | 0.0018 | (0.0065) | 0 | 0 | 0 | 0 | 0 | 0 |

TABLE 2
Mean (CV %) PK Parameters for Naltrexone Following Administration to Healthy Subjects.

| | | | 4 mg IN plus | | | | | |
|---|---|---|---|---|---|---|---|---|
| | | | 0.25% | | | | | |
| PK Parameter | 4 mg IN^{a} | | Intravail ® ^{b} | | 2 mg IM^{c} | | 50 mg Oral^{c} | |
| Cₘₐₓ (ng/mL) | 5.35 | (66.8) | 15.7 | (52.0) | 4.10 | (34.0) | 9.34 | (31.8) |
| Cₘₐₓ/Dose (ng/mL/mg) | 1.48 | (66.8) | 4.35 | (52.0) | 2.27 | (34.0) | 0.206 | (31.8) |
| Tₘₐₓ (h) ^{d} | 0.50 | (0.17, 2.00) | 0.17 | (0.083, 0.33) | 0.33 | (0.17, 1.00) | 0.50 | (0.33, 3.00) |
| AUC₀₋ₜ (h · ng/mL) | 11.9 | (34.1) | 18.3 | (31.2) | 12.1 | (25.5) | 26.5 | (32.3) |
| AUC₀₋ₜ/Dose (h · ng/mL/mg) | 3.28 | (34.1) | 5.07 | (31.2) | 6.71 | (25.5) | 0.587 | (32.3) |
| AUC_{0-inf} (h · ng/mL) | 12.0 | (33.7) | 18.5 | (31.0) | 12.3 | (25.6) | 26.9 | (31.8) |
| AUC_{0-inf}/Dose (h · ng/mL/mg) | 3.32 | (33.7) | 5.10 | (31.0) | 6.78 | (25.6) | 0.594 | (31.8) |
| AUCₑₓₜᵣₐₚ (%) | 1.09 | (57.0) | 0.707 | (44.0) | 1.01 | (71.7) | 1.38 | (70.1) |
| CL/F (L/h) | 330 | (28.9) | 214 | (33.6 | 154 | (19.0) | 1890 | (41.4) |
| λ_{z} (1/h) | 0.281 | (15.1) | 0.317 | (15.1) | 0.361 | (16.8) | 0.122 | (38.0) |
| t_{1/2} (h) | 2.52 | (14.9) | 2.23 | (14.9) | 1.97 | (15.5) | 6.41 | (36.6) |

| | | | | | | | |
|---|---|---|---|---|---|---|---|
| Fᵣₑₗ | 0.481 | (36.1)^{c} | 0.783 | (17.7)^{c} | NA | 0.0903 | (37.0) |

NA = Not applicable; Frel = Bioavailability relative to IM dose, calculated as ratio of AUC_{inf}/Dose for IN or PO route relative to IM route.
^{a}N = 13;
^{b} N = 12;
^{c}N = 10;
^{d} Median (minimum, maximum)

Following IN administration of 4 mg naltrexone, the mean concentration at 2.5 minutes postdose was 0.117 ng/mL. When 0.25% Intravail® was added to the formulation, the mean concentration was 10 times greater (1.15 ng/mL) at 2.5 minutes. At 5 minutes postdose, the mean concentrations of naltrexone with and without Intravail® were 11.9 ng/mL and 1.51 ng/mL, respectively, an 8-fold difference. The addition of 0.25% Intravail® to the IN formulation decreased median Tₘₐₓ from 30 minutes to 10 minutes and increased Cₘₐₓ almost 3-fold (15.7 versus 5.35 ng/mL). Overall exposure as measured by AUC_{0-inf} increased by 54%, indicating that the main effect of Intravail® was to increase the rate of absorption more than the extent.

The mean plasma concentrations of naltrexone at 2.5 and 5 minutes after administration of 2 mg naltrexone IM were 0.678 ng/mL and 1.04 ng/mL, respectively. The mean Cₘₐₓ value of 4.10 ng/mL 20 minutes after the 2 mg IM dose was 23% less than after the 4 mg IN dose and 74% less compared to when Intravail® was part of the IN formulation.

The mean Cₘₐₓ value after the oral dose was 9.34 ng/mL, which was less than observed after the IN dose with Intravail® even though 50 mg was administered orally compared to only 4 mg IN.

The mean terminal phase half-life (t_{1/2}) of naltrexone was 1.97 hours to 2.52 hours after IM and IN administration. The t_{1/2} was 6.41 hours after the oral dose.

When AUC_{0-inf} values were corrected for dose, the relative bioavailability of naltrexone after the IN doses with and without 0.25% Intravail® was 78% and 48%, respectively, compared to the IM administration. The relative bioavailability for the oral dose was only 9%, indicating extensive first pass metabolism by the gastrointestinal tract and liver.

Statistical analysis of dose-adjusted PK parameters suggested exposure for the IN dose was approximately 48% or 60% of the IM dose on a per mg basis, in terms of geometric least-squares mean (GM) dose-adjusted AUC and Cₘₐₓ, respectively. IN administration of naltrexone with 0.25% Intravail® resulted in dose-adjusted exposure that was higher than the IM route in terms of Cₘₐₓ (geometric least-squares mean ratio between treatments [GMR] of 188%) and lower in terms of AUC (GMR of 76%). For the oral route, the GMR for dose-adjusted naltrexone exposure was approximately 9% of the IM dose.

TABLE 3
Mixed-Effects ANOVA Results for Naltrexone Pharmacokinetic
Parameters Following Intranasal or Oral Administration
vs. Intramuscular Administration to Healthy Subjects.

| | | | 90% CI of GMR | |
|---|---|---|---|---|
| | Comparison | GMR | (%) | |
| PK Parameter | (2 mg IM Reference) | (%) | Lower | Upper |
| Cmax | 4 mg IN vs 2 mg IM | 121 | 91.1 | 160 |
| (ng/mL) | 4 mg IN plus Intravail ® | 377 | 321 | 442 |
| | vs 2 mg IM | | | |
| | 50 mg PO vs 2 mg IM | 231 | 190 | 282 |
| AUC0-t | 4 mg IN vs 2 mg IM | 96.4 | 82.6 | 112 |
| (h · ng/mL) | 4 mg IN plus Intravail ® | 152 | 136 | 169 |
| | vs 2 mg IM | | | |
| | 50 mg PO vs 2 mg IM | 221 | 182 | 268 |
| AUC0-inf | 4 mg IN vs 2 mg IM | 96.4 | 82.7 | 112 |
| (h · ng/mL) | 4 mg IN plus Intravail ® | 151 | 136 | 168 |
| | vs 2 mg IM | | | |
| | 50 mg PO vs 2 mg IM | 221 | 183 | 268 |
| Cmax/Dose | 4 mg IN vs 2 mg IM | 60.4 | 45.5 | 80.2 |
| (ng/mL/mg) | 4 mg IN plus Intravail ® | 188 | 161 | 221 |
| | vs 2 mg IM | | | |
| | 50 mg PO vs 2 mg IM | 9.3 | 7.6 | 11.3 |
| AUC0-t/Dose | 4 mg IN vs 2 mg IM | 48.2 | 41.3 | 56.2 |
| (h · ng/mL/mg) | 4 mg IN plus Intravail ® | 75.9 | 68.1 | 84.6 |
| | vs 2 mg IM | | | |
| | 50 mg PO vs 2 mg IM | 8.8 | 7.3 | 10.7 |
| AUC0-inf/Dose | 4 mg IN vs 2 mg IM | 48.2 | 41.4 | 56.2 |
| (h · ng/mL/mg) | 4 mg IN plus Intravail ® | 75.7 | 68 | 84.2 |
| | vs 2 mg IM | | | |
| | 50 mg PO vs 2 mg IM | 8.9 | 7.3 | 10.7 |

GMR = Geometric least-squares mean ratio between treatments (expressed as percentage of reference)

The mean Cₘₐₓ values of 6β-naltrexol were 1.5 ng/mL after the IM administration and approximately 3 ng/mL after the IN administration; Cmax was 90.7 ng/mL after the 50-mg oral dose (see Tables 2 and 3). When adjusted for the administered dose, the Cmax values were similar for the IN and IM doses (0.833 and 0.838 ng/mL/mg) but approximately 2-fold higher (2.00 ng/mL/mg) after oral administration.

Values of AUC_{0-inf} also were increased considerably after the oral dose in comparison to the IN and IM doses (675 h·ng/mL and 44.0 to 27.1 h·ng/mL, respectively). The greater extent of first pass metabolism of naltrexone was evident in the ratio of AUC_{0-inf} for 6β-naltrexol compared to that of naltrexone: after the IN and IM doses, the ratio was about 2.2 to 3.7, while it was 25 after the oral dose.

The mean t_{1/2} A of the metabolite was 12.4 to 13.9 hours and was independent of the route of administration.

Naltrexol Results.

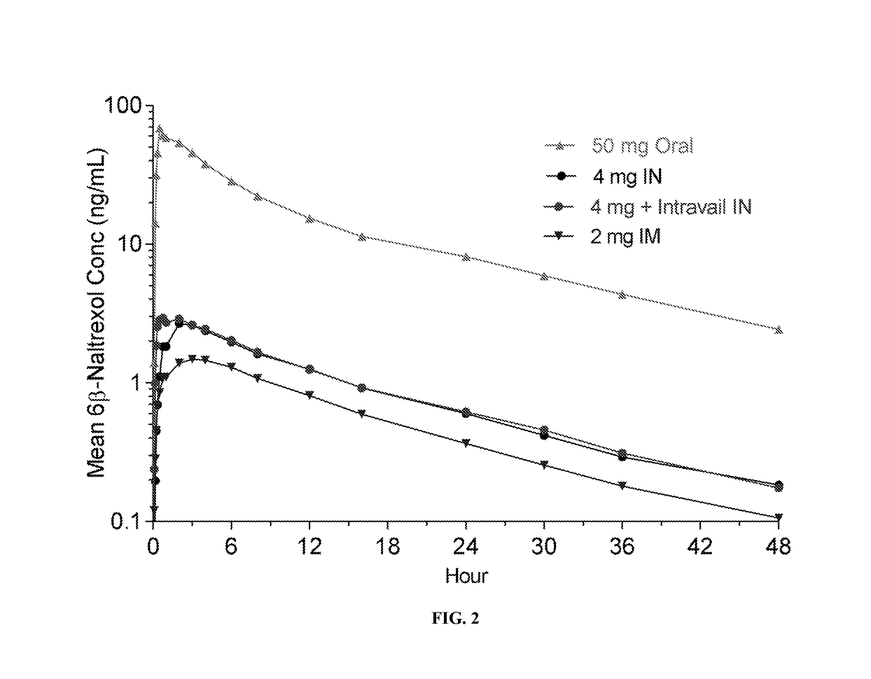

The results for naltrexol administration are shown below at Tables 4 and 5 and at FIG. 2.

TABLE 4
Mean (SD) concentrations of 6β-naltrexol following a single IN, IM or
oral administration to healthy subjects.

| | Treatment | | | | | | | |
|---|---|---|---|---|---|---|---|---|
| | | | 4 mg IN + 0.25% | | | | | |
| Hour | 4 mg IN | | Intravail ® | | 2 mg IN | | 50 mg Oral | |
| 0 | 0 | 0 | 0.0682 | (0.0257) | 0.0661 | (0.0256) | 0.0454 | (0.0141) |
| 0.042 | 0 | 0 | 0.082 | (0.0378) | 0.0627 | (0.0248) | 0.0448 | (0.0202) |
| 0.083 | 0.0321 | (0.0432) | 0.238 | (0.146) | 0.12 | (0.117) | 1.4 | (3.49) |
| 0.17 | 0.196 | (0.196) | 0.994 | (0.558) | 0.283 | (0.281) | 14.2 | (31) |
| 0.25 | 0.45 | (0.448) | 1.86 | (0.763) | 0.454 | (0.293) | 31.6 | (47.3) |
| 0.33 | 0.693 | (0.624) | 2.55 | (0.918) | 0.677 | (0.385) | 45.5 | (43.7) |
| 0.5 | 1.11 | (0.559) | 2.84 | (0.748) | 0.852 | (0.328) | 68.7 | (41.3) |
| 0.75 | 1.82 | (1.16) | 2.93 | (0.757) | 1.08 | (0.452) | 60.8 | (28.2) |
| 1 | 1.83 | (0.815) | 2.73 | (0.481) | 1.1 | (0.404) | 58.6 | (18.2) |
| 2 | 2.68 | (0.842) | 2.9 | (0.767) | 1.39 | (0.462) | 54 | (16.3) |
| 3 | 2.61 | (0.793) | 2.61 | (0.708) | 1.48 | (0.402) | 45.8 | (15.2) |
| 4 | 2.37 | (0.669) | 2.45 | (0.598) | 1.46 | (0.388) | 38 | (12.2) |
| 6 | 1.97 | (0.554) | 2.03 | (0.399) | 1.3 | (0.252) | 28.5 | (7.52) |
| 8 | 1.62 | (0.418) | 1.67 | (0.27) | 1.08 | (0.165) | 22.2 | (5.51) |
| 12 | 1.26 | (0.299) | 1.25 | (0.176) | 0.81 | (0.131) | 15.4 | (2.85) |
| 16 | 0.919 | (0.229) | 0.923 | (0.155) | 0.595 | (0.115) | 11.4 | (1.78) |
| 24 | 0.602 | (0.194) | 0.618 | (0.145) | 0.365 | (0.0808) | 8.14 | (2.09) |
| 30 | 0.418 | (0.114) | 0.457 | (0.116) | 0.255 | (0.0666) | 5.93 | (1.85) |
| 36 | 0.292 | (0.0868) | 0.312 | (0.0862) | 0.18 | (0.0478) | 4.35 | (1.54) |
| 48 | 0.184 | (0.0645) | 0.175 | (0.0623) | 0.106 | (0.0329) | 2.43 | (0.882) |

TABLE 5
Mean (CV %) PK Parameters for 6β-Naltrexol Following Administration to Healthy Subjects.

| | | | 4 mg IN plus | | | | | |
|---|---|---|---|---|---|---|---|---|
| | | | 0.25% | | | | | |
| PK Parameter | 4 mg IN^{a} | | Intravail ® ^{b} | | 2 mg IM^{c} | | 50 mg Oral^{c} | |
| Cₘₐₓ (ng/mL) | 3.01 | (33.2) | 3.29 | (23.7) | 1.52 | (26.8) | 90.7 | (30.3) |
| Cₘₐₓ/Dose (ng/mL/mg) | 0.833 | (33.2) | 0.908 | (23.7) | 0.838 | (26.8) | 2.00 | (30.3) |
| Tₘₐₓ (h) ^{d} | 2.00 | (0.75, 6.0) | 0.75 | (0.25, 4.00) | 3.00 | (0.75, 4.00) | 0.63 | (0.25, 3.00) |
| AUC₀₋ₜ (h · ng/mL) | 40.3 | (23.3) | 43.0 | (17.4) | 25.1 | (18.3) | 614 | (19.5) |
| AUC₀₋ₜ/Dose | 11.1 | (23.3) | 11.9 | (17.4) | 13.9 | (18.3) | 13.6 | (19.5) |
| (h · ng/mL/mg) | | | | | | | | |
| AUC_{0-inf} (h · ng/mL) | 44.0 | (23.1) | 46.3 | (18.3) | 27.1 | (19.0) | 675 | (19.9) |
| AUC_{0-inf}/Dose | 12.2 | (23.1) | 12.8 | (18.3) | 15.0 | (19.0) | 14.9 | (19.9) |
| (h · ng/mL/mg) | | | | | | | | |
| AUCₑₓₜᵣₐₚ (%) | 8.57 | (46.1) | 7.02 | (37.3) | 7.02 | (29.4) | 8.79 | (57.3) |
| λ_{z} (1/h) | 0.0530 | (21.8) | 0.0553 | (15.1) | 0.0570 | (14.8) | 0.0510 | (15.8) |
| t_{1/2} (h) | 13.7 | (22.7) | 12.8 | (14.6) | 12.4 | (13.2) | 13.9 | (15.9) |

*Median (Min, Max) statistics presented for Tmax. All other values presented as: Mean (Percent coefficient of variation);
^{a}N = 13;
^{b} N = 12;
^{c}N = 10;
^{d} Median (minimum, maximum)

Except for the mean Cₘₐₓ of naltrexone following the 4 mg IN dose, which was approximately 2-fold higher in females compared to males, there was no clinically meaningful difference between the sexes for the PK parameters of either naltrexone or 60-naltrexol following IN, IM, or PO administration.

Safety.

In total, 10 of 14 subjects (71%) in the safety population experienced at least one AE (any dosing period, any relationship to drug). The most frequent AEs were of the Nervous System Disorders in the system, organ, class (SOC) designation (7 subjects, 50%), and dizziness was the most frequent AE regardless of severity or attribution (5 subjects, 36%). No severe AEs were observed, and only one moderate AE was observed, a case of dizziness after the first dose (4 mg IN) that was considered related to the study agent. Three subjects experienced AEs that were unexpected (“unexpected adverse event” or “UAE,” defined as an AE that was not described with respect to nature, severity, or frequency in the current product package insert). Two UAEs were considered unrelated to the study agent and one treatment-related UAE of mild syncope after administration of the Day 1 dose (4 mg IN). Three subjects were discontinued from the study due to AEs (hypertension, syncope, and out-of-range pre-dose vital signs).

Results above suggest that IN naltrexone enters the plasma more quickly than oral naltrexone, and achieves a Tₘₐₓ that is higher than intramuscular naltrexone and lower than oral naltrexone. Additionally, the data suggests that administration of IN naltrexone avoids elevated levels of the metabolite 6β-naltrexol.

### Formulations of Intranasal Naltrexone

The following tables set forth examples of formulations of naltrexone for intranasal administration for the treatment of disorders disclosed herein. Table 6 sets forth simple aqueous solution formulations such as those used in the experiment above, to be dispensed in increments of about 100 μL.

TABLE 6

| | Naltrexone | | | |
|---|---|---|---|---|
| | HCl, dose | Absorption | μL per | Conc., |
| Ex. | (mg) | Enhancer | dose | mg/mL |
| 1 | 1 | n/a | 100 | 10 |
| 2 | 1 | Intravail 0.25% | 100 | 10 |
| 3 | 2 | n/a | 100 | 20 |
| 4 | 2 | Intravail 0.25% | 100 | 20 |
| 5 | 3 | n/a | 100 | 30 |
| 6 | 3 | Intravail 0.25% | 100 | 30 |
| 7 | 4 | n/a | 100 | 40 |
| 8 | 4 | Intravail 0.25% | 100 | 40 |
| 9 | 5 | n/a | 100 | 50 |
| 10 | 5 | Intravail 0.25% | 100 | 50 |
| 11 | 6 | n/a | 100 | 60 |
| 12 | 6 | Intravail 0.25% | 100 | 60 |
| 13 | 7 | n/a | 100 | 70 |
| 14 | 7 | Intravail 0.25% | 100 | 70 |
| 15 | 8 | n/a | 100 | 80 |
| 16 | 8 | Intravail 0.25% | 100 | 80 |

Table 7 sets forth formulations for intranasal administration in 100 μL of an aqueous solution including excipients such as an isotonicity agent, a stabilizing agent, and/or a compound which acts as a preservative or surfactant. EDTA stands for disodium edetate and BZK stands for benzalkonium chloride.

TABLE 7

| | Nal- | | | | |
|---|---|---|---|---|---|
| | trex- | | | | Preserva- |
| | one | Absorption | Isotonicity | Stabilizing | tive/Sur- |
| Ex. | HCl | Enhancer | Agent | Agent | factant |
| 17 | 2 mg | n/a | NaCl 0.74% | n/a | n/a |
| 18 | 2 mg | n/a | NaCl 0.74% | EDTA 0.2% | n/a |
| 19 | 2 mg | n/a | NaCl 0.74% | n/a | BZK 0.01% |
| 20 | 2 mg | n/a | NaCl 0.74% | EDTA 0.2% | BZK 0.01% |
| 21 | 4 mg | n/a | NaCl 0.74% | n/a | n/a |
| 22 | 4 mg | n/a | NaCl 0.74% | EDTA 0.2% | n/a |
| 23 | 4 mg | n/a | NaCl 0.74% | n/a | BZK 0.01% |
| 24 | 4 mg | n/a | NaCl 0.74% | EDTA 0.2% | BZK 0.01% |
| 25 | 2 mg | Intravail 0.25% | NaCl 0.74% | n/a | n/a |
| 26 | 2 mg | Intravail 0.25% | NaCl 0.74% | EDTA 0.2% | n/a |
| 27 | 2 mg | Intravail 0.25% | NaCl 0.74% | n/a | BZK 0.01% |
| 28 | 2 mg | Intravail 0.25% | NaCl 0.74% | EDTA 0.2% | BZK 0.01% |
| 29 | 4 mg | Intravail 0.25% | NaCl 0.74% | n/a | n/a |
| 30 | 4 mg | Intravail 0.25% | NaCl 0.74% | EDTA 0.2% | n/a |
| 31 | 4 mg | Intravail 0.25% | NaCl 0.74% | n/a | BZK 0.01% |
| 32 | 4 mg | Intravail 0.25% | NaCl 0.74% | EDTA 0.2% | BZK 0.01% |
| 33 | 2 mg | Intravail 0.18% | NaCl 0.74% | n/a | n/a |
| 34 | 2 mg | Intravail 0.18% | NaCl 0.74% | EDTA 0.2% | n/a |
| 35 | 2 mg | Intravail 0.18% | NaCl 0.74% | n/a | BZK 0.01% |
| 36 | 2 mg | Intravail 0.18% | NaCl 0.74% | EDTA 0.2% | BZK 0.01% |
| 37 | 4 mg | Intravail 0.18% | NaCl 0.74% | n/a | n/a |
| 38 | 4 mg | Intravail 0.18% | NaCl 0.74% | EDTA 0.2% | n/a |
| 39 | 4 mg | Intravail 0.18% | NaCl 0.74% | n/a | BZK 0.01% |
| 40 | 4 mg | Intravail 0.18% | NaCl 0.74% | EDTA 0.2% | BZK 0.01% |
| 41 | 2 mg | Benzalkonium | NaCl 0.74% | n/a | n/a |
| | | chloride, 0.01% | | | |
| 42 | 2 mg | Benzalkonium | NaCl 0.74% | EDTA 0.2% | n/a |
| | | chloride, 0.01% | | | |
| 43 | 2 mg | Benzalkonium | NaCl 0.74% | n/a | BZK 0.01% |
| | | chloride, 0.01% | | | |
| 44 | 2 mg | Benzalkonium | NaCl 0.74% | EDTA 0.2% | BZK 0.01% |
| | | chloride, 0.01% | | | |
| 45 | 4 mg | Benzalkonium | NaCl 0.74% | n/a | n/a |
| | | chloride, 0.01% | | | |
| 46 | 4 mg | Benzalkonium | NaCl 0.74% | EDTA 0.2% | n/a |
| | | chloride, 0.01% | | | |
| 47 | 4 mg | Benzalkonium | NaCl 0.74% | n/a | BZK 0.01% |
| | | chloride, 0.01% | | | |
| 48 | 4 mg | Benzalkonium | NaCl 0.74% | EDTA 0.2% | BZK 0.01% |
| | | chloride, 0.01% | | | |

Also provided are examples 1-48A which additionally contain an amount of hydrochloric acid sufficient to achieve a pH of 3.5-5.5. The acid should be pharmaceutically acceptable, for example, hydrochloric acid.

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this invention, and without departing from the spirit and scope thereof, can make various changes and modifications of the invention to adapt it to various usages and conditions.

Although the present invention has been described with reference to specific details of certain embodiments thereof in the above examples, it will be understood that modifications and variations are encompassed within the spirit and scope of the invention.

What is claimed is:

1. A method of inhibiting or reducing opioid overdose risk in a subject at risk for opioid overdose, comprising administering to the subject an intranasal formulation comprising a therapeutically effective amount of naltrexone, thereby inhibiting or reducing opioid overdose risk in the subject.

2. The method of claim 1, wherein each dose of the composition comprises between about 2 and about 12 mg naltrexone or a salt thereof.

3. The method of claim 2, the intranasal formulation further comprising an excipient.

4. The method of claim 3, wherein the excipient is an absorption enhancer.

5. The method of claim 1, wherein the intranasal formulation is an aqueous solution.

6. The method of claim 1, wherein the intranasal formulation comprises about 4 mg naltrexone or a salt thereof.

7. The method of claim 1, wherein about 0.1 mL of the nasal formulation is delivered to the subject.

8. The method of claim 1, wherein the intranasal formulation is at a concentration of about 40 mg/mL.

9. The method of claim 1, wherein the intranasal formulation is administered as a single administration to one nostril.

10. The method of claim 1, wherein the intranasal formulation is administered as two administrations, one to each nostril.

11. The method of claim 1, the formulation further comprising an amount of acid sufficient to achieve a pH between 3.5 and 5.5.

12. The method of claim 1, wherein administering the intranasal formulation provides a maximum plasma concentration (Cₘₐₓ) ranging between about 4 ng/mL and about 6 4 ng/mL, a time to maximum plasma concentration (Tₘₐₓ) under 20 minutes, or both.

13. A method of achieving plasma levels of a drug therapeutically effective to reverse narcotic depression, reduce or inhibit the desire to consume (or otherwise administer) a substance which produces reward, or reduce or inhibit the desire to engage in a behavior which produces reward, comprising administering to the subject an intranasal formulation comprising a therapeutically effective amount of naltrexone or a pharmaceutically acceptable salt thereof, thereby achieving plasma levels of the drug therapeutically effective to reverse narcotic depression, reduce or inhibit the desire to consume (or otherwise administer) a substance which produces reward, or reduce or inhibit the desire to engage in a behavior which produces reward.

14. The method of claim 13, the intranasal formulation further comprising an excipient.

15. The method of claim 14, wherein the excipient is an absorption enhancer.

16. The method of claim 13, wherein the intranasal formulation comprises an aqueous solution.

17. The method of claim 13, wherein the intranasal formulation comprises about 4 mg naltrexone or a salt thereof.

18. The method of claim 13, wherein about 0.1 mL of the intranasal formulation is delivered to the subject.

19. The method of claim 13, wherein the intranasal formulation is at a concentration of about 40 mg/mL.

20. The method of claim 13, wherein the intranasal formulation is administered as a single administration to one nostril.

21. The method of claim 13, wherein the intranasal formulation is administered as two administrations, one to each nostril.

22. The method of claim 13, the formulation further comprising an amount of acid sufficient to achieve a pH between 3.5 and 5.5.

23. The method of claim 13, wherein the intranasal formulation comprising naltrexone is administered prior to consuming (or otherwise administering) a substance which produces reward, or engaging in a behavior which produces reward.

24. The method of claim 13, wherein the intranasal formulation comprising naltrexone is administered contemporaneously with consuming (or otherwise administering) a substance which produces reward, or engaging in a behavior which produces reward.

25. The method of claim 13, wherein the therapeutically effective amount comprises from about 4 mg to about 16 mg of naltrexone per day.

26. The method of claim 13, wherein the therapeutically effective amount of naltrexone is administered in 4-mg doses throughout the day as needed by the subject.

27. The method of claim 13, wherein the therapeutically effective amount of naltrexone is administered as a first 4-mg dose in the morning, and subsequent 4-mg doses as needed prior to consuming (or otherwise administering) a substance which produces reward, or engaging in a behavior which produces reward.

28. A pharmaceutical formulation for intranasal administration comprising, in an aqueous solution of between about 50 μL and about 250 μL:
between about 2 mg and about 12 mg naltrexone hydrochloride or a hydrate thereof; and
between about 0.2 to about 2.0 mg of an isotonicity agent.

29. The pharmaceutical formulation of claim 28, comprising about 4 mg naltrexone hydrochloride.

30. The pharmaceutical formulation of claim 28, comprising between about 0.005 mg and about 2.5 mg of a compound which is at least one of a preservative, a cationic surfactant, and an absorption enhancer.

31. The pharmaceutical formulation of claim 28, comprising between about 0.1 mg and about 0.5 mg of a stabilizing agent.

32. The pharmaceutical formulation of claim 28, comprising an amount of an acid sufficient to achieve a pH between 3.5 and 5.5.

33. The pharmaceutical formulation of claim 28, comprising:
between about 0.1 mg and about 0.5 mg stabilizing agent; and
an amount of an acid sufficient to achieve a pH between 3.5 and 5.5.

34. The pharmaceutical formulation of claim 28, wherein the isotonicity agent is sodium chloride.

35. The pharmaceutical formulation of claim 28, further comprising an excipient.

36. The pharmaceutical formulation of claim 35, wherein the excipient is an absorption enhancer.

37. The pharmaceutical formulation of claim 30, wherein the compound which is at least one of the preservative, the cationic surfactant, and the absorption enhancer is chosen from benzalkonium chloride and an alkylsaccharide.

38. The pharmaceutical formulation of claim 33, wherein the stabilizing agent is disodium edetate.

39. The pharmaceutical formulation of claim 33, wherein the acid is hydrochloric acid.

40. The pharmaceutical formulation of claim 33, wherein the isotonicity agent is sodium chloride, wherein the compound which is at least one of the preservative, the cationic surfactant, and the absorption enhancer is chosen from benzalkonium chloride and an alkylsaccharide, wherein the stabilizing agent is disodium edetate, and wherein the acid is hydrochloric acid.

41. The pharmaceutical formulation of claim 28, in an aqueous solution of about 100 μL comprising:
about 4 mg of naltrexone hydrochloride;
about 0.74 mg sodium chloride;
about 0.01 mg benzalkonium chloride or about 0.25% dodecyl maltoside;
about 0.2 mg disodium edetate; and
an amount of hydrochloric acid sufficient to achieve a pH between 3.5 and 5.5.

42. A method for treating or preventing an opioid receptor-mediated, reward-based disease, disorder, addiction, or condition in a subject, comprising administering to the subject an intranasal formulation comprising a therapeutically effective amount of naltrexone or a pharmaceutically acceptable salt thereof, thereby treating or preventing the opioid receptor-mediated, reward-based disease, disorder, addiction, or condition in the subject.

43. The method of claim 42, wherein the disease, disorder, addiction, or condition is chosen from alcohol use disorder, tobacco use disorder, opioid use disorder, prescription drug use disorder, cocaine use disorder, cannabis use disorder, amphetamine use disorder, hallucinogen use disorder, inhalants use disorder, phencyclidine use disorder, kleptomania, pyromania, gambling, bulimia, and binge eating.

44. The method of claim 42, wherein the intranasal formulation is administered prior to exposure to an addictive substance or behavior.

45. The method of claim 42, wherein the intranasal formulation is administered between about 1 hour and about 2 hours prior to exposure to an addictive substance or behavior.

46. The method of claim 42, wherein the intranasal formulation is administered contemporaneously with exposure to an addictive substance or behavior.

47. The method of claim 42, wherein the intranasal formulation comprises an aqueous solution.

48. The method of claim 42, wherein the intranasal formulation comprises about 4 mg naltrexone or a salt thereof.

49. The method of claim 42, wherein about 0.1 mL of the intranasal formulation is delivered to the subject.

50. The method of claim 42, wherein said formulation is at a concentration of about 40 mg/mL.

51. The method of claim 42, wherein each dose of the intranasal formulation is administered as a single administration to one nostril.

52. The method of claim 42, wherein each dose of the intranasal formulation is administered as two administrations, one to each nostril.

53. The method of claim 42, wherein the intranasal formulation further comprises a compound chosen from benzalkonium chloride, alkylsaccharides, chitosans, cyclodextrins, deoxycholic acid, glycocholic acid, laureth-9, taurocholic acid, and taurodihydrofusidic acid.

54. The method of claim 42, wherein the therapeutically effective amount is between about 4 mg and about 16 mg of naltrexone per day.

55. The method of claim 54, wherein the therapeutically effective amount of naltrexone is administered in 4 mg doses throughout the day as needed by the subject.

56. The method of claim 42, wherein the therapeutically effective amount of naltrexone is administered as a first 4 mg dose in the morning, and subsequent 4 mg doses as needed prior to exposure to an addictive substance or behavior.

57. A method for achieving plasma levels of a drug therapeutically effective to reverse narcotic depression in less than 15 minutes, comprising administering to the subject an intranasal formulation comprising a therapeutically effective amount of naltrexone or a pharmaceutically acceptable salt thereof.

58. The method of claim 57, wherein the excipient is chosen from benzalkonium chloride, chitosans, cyclodextrins, deoxycholic acid, glycocholic acid, laureth-9, taurocholic acid, and taurodihydrofusidic acid.

59. The method of claim 57, wherein the intranasal formulation comprises an aqueous solution.

60. The method of claim 57, wherein the intranasal formulation comprises about 4 mg naltrexone or a salt thereof.

61. The method of claim 57, wherein about 0.1 mL of said formulation is delivered to the subject.

62. The method of claim 57, wherein said formulation is at a concentration of about 40 mg/mL.

63. The method of claim 57, wherein the intranasal formulation is administered as a single administration to one nostril.

64. The method of claim 57, wherein the intranasal formulation is administered as two administrations, one to each nostril.

65. The method of claim 57, further comprising an amount of acid sufficient to achieve a pH between 3.5 and 5.5.

66. A method of achieving a plasma concentration of naltrexone therapeutically effective to treat opioid overdose in a patient in need thereof while maintaining a plasma concentration of 6β-naltrexol below about 4 ng/mL, comprising the intranasal administration of a pharmaceutical formulation comprising between about 2 mg and about 16 mg naltrexone or a salt or hydrate thereof.

67. The method of claim 66, wherein the naltrexone is naltrexone hydrochloride.

68. The method of claim 67, wherein the pharmaceutical formulation comprises about 4 mg naltrexone hydrochloride.

69. The method of claim 66, wherein the pharmaceutical formulation is an aqueous solution.

70. The method of claim 69, wherein the aqueous solution has a volume between about 50 μL and about 250 μL per dose.

71. The method of claim 70, wherein about 0.1 mL of the pharmaceutical formulation is delivered to the subject.

72. The method of claim 70, wherein the pharmaceutical formulation is at a concentration of about 40 mg/mL naltrexone.

73. The method of claim 70, wherein the pharmaceutical formulation is administered as a single administration to one nostril.

74. The method of claim 70, wherein the pharmaceutical formulation is administered as two administrations, one to each nostril.

75. The method of claim 70, wherein the pharmaceutical formulation comprises between about 0.005 mg and about 2.5 mg of a compound which is at least one of a preservative, a cationic surfactant, and an excipient.

76. The method of claim 70, wherein the pharmaceutical formulation comprises between about 0.1 mg and about 0.5 mg of a stabilizing agent.

77. The method of claim 70, wherein the pharmaceutical formulation comprises an amount of an acid sufficient to achieve a pH between 3.5 and 5.5.

78. The method of claim 70, wherein the pharmaceutical formulation comprises:
between about 0.1 mg and about 0.5 mg of a stabilizing agent; and
an amount of an acid sufficient to achieve a pH between 3.5 and 5.5.

79. The method of claim 70, wherein the isotonicity agent is NaCl.

80. The method of claim 70, wherein the pharmaceutical formulation comprises an absorption enhancer.

81. A method of treating a reward based disorder in a subject within 40 minutes of administration of an intranasal pharmaceutical formulation, the formulation comprising between about 2 mg and about 12 mg naltrexone and between about 0.01% and about 2.5% (w/v) dodecyl maltoside, thereby treating the reward based disorder in the subject.

82. The method of claim 81, wherein the reward-based disorder is abuse of an addictive substance.

83. The method of claim 81, wherein the reward based disorder is a compulsive behavior chosen from bulimia nervosa, binge eating, sex disorder, and pathological gambling.

84. The method of claim 81, wherein the subject suffers from obsessive compulsive disorder (OCD), and the method treats the compulsive behavior of the OCD.

85. The method of claim 81, wherein the reward-based disorder is chosen from alcohol use disorder, tobacco use disorder, opioid use disorder, prescription drug use disorder, cocaine use disorder, cannabis use disorder, amphetamine use disorder, hallucinogen use disorder, inhalants use disorder, phencyclidine use disorder, kleptomania, pyromania, pathological gambling, bulimia nervosa, and binge eating.

86. The method of claim 81, wherein the reward-based disorder is reduced within 15 minutes of administration.

87. The method of claim 86, wherein the reward-based disorder is reduced within 8 minutes of administration.

88. The method of claim 81, wherein the intranasal formulation is administered prior to exposure to an addictive substance or behavior.

89. The method of claim 81, wherein the intranasal formulation is administered between about 1 minutes and about 30 minutes prior to exposure to the addictive substance or engagement in the behavior that is associated with the reward-based disorder.

90. The method of claim 81, wherein the intranasal formulation is administered contemporaneously with exposure to the addictive substance or engagement in the behavior that is associated with the reward-based disorder.

91. The method of claim 81, wherein the intranasal formulation comprises an aqueous solution.

92. The method of claim 81, wherein the intranasal formulation comprises about 4 mg naltrexone or a salt thereof.

93. The method of claim 81, wherein the intranasal formulation comprises between about 0.1% and about 0.5% (w/v) dodecyl maltoside.

94. The method of claim 93, wherein the intranasal formulation comprises between about 0.05% and about 1.5% (w/v) dodecyl maltoside, or between about 0.15% and about 0.35% (w/v) dodecyl maltoside.

95. The method of claim 81, wherein about 0.1 mL of the intranasal formulation is delivered to the subject.

96. The method of claim 81, wherein the intranasal formulation is at a concentration of 30 mg/mL.

97. The method of claim 81, wherein each dose of the intranasal formulation is administered as a single administration to one nostril.

98. The method of claim 81, wherein each dose of the intranasal formulation is administered as two administrations, one to each nostril.

99. The method of claim 81, wherein a therapeutically effective amount is between about 4 mg and about 16 mg of naltrexone per day.

100. The method of claim 99, wherein the therapeutically effective amount of naltrexone is administered in 4 mg doses throughout the day as needed by the subject.

101. The method of claim 81, wherein a therapeutically effective amount of naltrexone is administered as a first 4 mg dose in the morning, and subsequent 4 mg doses as needed prior to exposure to the addictive substance or engagement in the behavior that is associated with the reward-based disorder.

102. A method of treating a reward based disorder in a patient for at least 2 hours, comprising the administration of an intranasal pharmaceutical formulation, the formulation comprising between about 2 mg and about 12 mg naltrexone and between about 0.01% and about 2.5% (w/v) dodecyl maltoside, thereby treating the reward-based disorder in the patient.

103. The method of claim 102, wherein the reward-based disorder is abuse of an addictive substance.

104. The method of claim 102, wherein the reward-based disorder is a compulsive behavior chosen from binge eating, sexual use disorder, gambling, and risk-taking.

105. The method of claim 102, wherein the subject suffers from obsessive compulsive disorder (OCD), and the method treats the compulsive behavior of the OCD.

106. The method of claim 102, wherein the reward-based disorder is chosen from alcohol use disorder, tobacco use disorder, opioid use disorder, prescription drug use disorder, cocaine use disorder, cannabis use disorder, amphetamine use disorder, hallucinogen use disorder, inhalants use disorder, phencyclidine use disorder, kleptomania, pyromania, gambling, bulimia, and binge eating.

107. The method of claim 102, wherein the reward-based disorder is reduced within 15 minutes of administration.

108. The method of claim 107, wherein the reward-based disorder is reduced within 8 minutes of administration.

109. The method of claim 102, wherein the intranasal formulation is administered between about 1 hour and about 2 hours prior to exposure to the addictive substance or engagement in the behavior that is associated with the reward-based disorder.

110. The method of claim 102, wherein the intranasal formulation is administered between about 10 minutes and about 30 minutes prior to exposure to the addictive substance or engagement in the behavior that is associated with the reward-based disorder.

111. The method of claim 102, wherein the intranasal formulation is administered contemporaneously with exposure to the addictive substance or engagement in the behavior that is associated with the reward-based disorder.

112. The method of claim 102, wherein the intranasal formulation comprises an aqueous solution.

113. The method of claim 102, wherein the intranasal formulation comprises about 4 mg naltrexone or a salt thereof.

114. The method of claim 102, wherein the intranasal formulation comprises between about 0.1% and about 0.5% (w/v) dodecyl maltoside.

115. The method of claim 114, wherein the intranasal formulation comprises between about 0.15% and about 0.35% (w/v) dodecyl maltoside.

116. The method of claim 102, wherein about 0.1 mL of the intranasal formulation is delivered to the subject.

117. The method of claim 102, wherein the intranasal formulation is at a concentration of 30 mg/mL.

118. The method of claim 102, wherein each dose of the intranasal formulation is administered as a single administration to one nostril.

119. The method of claim 102, wherein each dose of the intranasal formulation is administered as two administrations, one to each nostril.

120. The method of claim 102, wherein a therapeutically effective amount is between about 4 mg and about 16 mg of naltrexone per day.

121. The method of claim 120, wherein the therapeutically effective amount of naltrexone is administered in 4 mg doses throughout the day as needed by the subject.

122. The method of claim 102, wherein a therapeutically effective amount of naltrexone is administered as a first 4 mg dose in the morning, and subsequent 4 mg doses as needed prior to exposure to the addictive substance or engagement in the behavior that is associated with the reward-based disorder.

123. An intranasal pharmaceutical formulation, comprising naltrexone that achieves a Cₘₐₓ of at least 5 ng/mL within 40 minutes.

124. The intranasal pharmaceutical formulation of claim 123, wherein the Cₘₐₓ is at least 15 ng/mL.

125. The intranasal pharmaceutical formulation of claim 123, wherein the Cₘₐₓ is achieved within 15 minutes of administration.

126. The intranasal pharmaceutical formulation of claim 125, wherein the Cₘₐₓ is achieved within 8 minutes of administration.

127. The intranasal pharmaceutical formulation of claim 123, comprising between about 2 mg and about 12 mg naltrexone hydrochloride or a hydrate thereof.

128. The intranasal pharmaceutical formulation of claim 127, comprising about 4 mg naltrexone hydrochloride.

129. The intranasal pharmaceutical formulation of claim 123, comprising between about 0.2 and about 2.0 mg of an isotonicity agent.

130. The intranasal pharmaceutical formulation of claim 123, between about 0.01% and about 2.5% (w/v) dodecyl maltoside.

131. The intranasal pharmaceutical formulation of claim 130, comprising between about 0.1% and about 0.5% (w/v) dodecyl maltoside.

132. The intranasal pharmaceutical formulation of claim 131, comprising between about 0.15% and about 0.35% (w/v) dodecyl maltoside.

133. The intranasal pharmaceutical formulation of claim 123, comprising between about 0.005 mg and about 0.015 mg of a compound which is at least one of a preservative, a cationic surfactant, and an absorption enhancer.

134. The intranasal pharmaceutical formulation of claim 123, comprising between about 0.1 mg and about 0.5 mg of a stabilizing agent.

135. The intranasal pharmaceutical formulation of claim 123, comprising an amount of an acid sufficient to achieve a pH between 3.5 and 5.5.

136. The intranasal pharmaceutical formulation of claim 123, comprising:
between about 0.1 mg and about 0.5 mg stabilizing agent; and
an amount of an acid sufficient to achieve a pH between 3.5 and 5.5.

137. The intranasal pharmaceutical formulation of claim 129, wherein the isotonicity agent is sodium chloride.

138. The intranasal pharmaceutical formulation of claim 133, wherein the compound which is at least one of the preservative, the cationic surfactant, and the absorption enhancer is benzalkonium chloride.

139. The intranasal pharmaceutical formulation of claim 134, wherein the stabilizing agent is disodium edetate.

140. The intranasal pharmaceutical formulation of claim 125, wherein the acid is hydrochloric acid.

141. The intranasal pharmaceutical formulation of claim 123, comprising sodium chloride, benzalkonium chloride, disodium edetate, and hydrochloric acid.

142. The intranasal pharmaceutical formulation of claim 123, in an aqueous solution of about 100 μL comprising:
about 4 mg of naltrexone hydrochloride;
about 0.74 mg sodium chloride;
about 0.01 mg benzalkonium chloride;
about 0.2 mg disodium edetate; and
an amount of hydrochloric acid sufficient to achieve a pH between 3.5 and 5.5.
